(19) United States
(12) Patent Application Publication
(10) Pub. No.: US 2024/0076380 A1
(43) Pub. Date: Mar. 7, 2024

(54) DEVICE AND METHODS FOR RAPIDLY SCREENING AND TREATING CLINICAL CANCER PATIENTS WITH IMMUNOREGULATORY DRUGS

(71) Applicant: Shanghai Lide Biotech Co., Ltd., Shanghai (CN)

(72) Inventors: Bin Xie, Shanghai (CN); Danyi Wen, Shanghai (CN)

(73) Assignee: Shanghai Lide Biotech Co., Ltd., Shanghai (CN)

(21) Appl. No.: 18/455,557

(22) Filed: Aug. 24, 2023

(30) Foreign Application Priority Data
Sep. 1, 2022 (CN) 202211066571.6

(51) Int. Cl. C07K 16/28; A61K 45/06; A61P 35/00; C12N 5/0783

(52) U.S. Cl. CPC C07K 16/2827; A61K 45/06; A61P 35/00; C07K 16/2818; C12N 5/0636

(57) ABSTRACT

The disclosure provides methods for rapid screening of the efficacy of immunoregulatory drugs, especially for cancer drug screening and treatment. The methods comprise an animal model wherein polymeric capsule tubes contain cells comprising tumor cells and immune cells in a single cell suspension. The capsule device is implanted into a mouse that is treated with an anti-cancer drug, especially an immunomodulatory anti-cancer drug. The methods provide a rapid and effective way for determining a specific individual's clinical response to an immunomodulatory drug with extremely high speed and subsequent efficacious treatment in the clinical setting.

This application filed under 35 U.S.C. §. 111(a) claims the priority benefit under 35 U.S.C. § 119 to Patent Application no. 202211066571.6, filed Sep. 1, 2022 with China's National Intellectual Property Administration, the application's content of which is fully incorporated by reference herein.

The invention relates to the field of biomedicine, in particular, to a method for rapid screening the efficacy of immunoregulating drugs and the use of such immunotherapy drugs to treat cancer in humans.

### BACKGROUND

Currently, many drugs with immune targeting and regulatory functions have been developed and applied to the treatment of numerous clinical diseases. In oncology, immunomodulatory drugs do not kill the tumor, but instead activate, recruit and derepress tumor specific lymphocytes to kill or suppress the tumor. CTLA4 antibody (ipilumumab) and PD1 antibody (Keytruda, Opdivo) are T cell checkpoint inhibitors that are used in the treatment of tumors such as melanoma, non-small cell lung cancer, small cell lung cancer, as well as head and neck squamous cell carcinoma.

However, it is impossible to accurately predict whether any immunomodulatory drug or combination of drugs will cause a positive clinical response, much less which drug or combination will provide the best possible clinical response. Moreover, it is critical to predict the effectiveness of a drug rapidly, since patients often do not have significant survival time to undergo trial and error testing of various treatments. As such, there is a great need for a screening method for immunomodulatory drugs that is both fast and accurate for use in clinical immunotherapy.

Currently, clinical diagnosis and treatment require a lengthy time to test for guidance on treatment regimen. For example, the classic humanized mouse patient derived xenografts experiment for the modeling of immunological treatments not only takes 2-5 months, but it also fails to meet the diversified needs of clinical patients for the selection of multiple immunomodulatory drugs. This development time also delays the process of new drug development. Therefore, there is a need for rapid and effective functional screening methods that allow precise treatment of patients along with correlative identification of biomarkers for different patient populations.

The current technical solutions to drug screening disclose drug sensitivity of tumor cells in vivo for chemotherapeutic drugs but do not provide guidance for screening immunoregulatory drugs. Indeed, classical screening methods all examine the effect of chemotherapeutic drugs or tumor-targeted drugs on primary and cultured tumor cells (U.S. Ser. No. 00/567,6924 A, 1997; U.S. Ser. No. 00/569,8413 A, 1997; CN108351348 A, 2017). Consequently, the methods taught in the prior art are incapable of rapidly screening the effect of immunomodulatory or quickly determining drug efficacy.

### SUMMARY OF THE INVENTION

The problem solved by the present invention is that it provides a method for rapidly screening the efficacy of immunomodulatory drugs, as well as identifying immunomodulatory drugs and combinations thereof that will be successful clinical cancer treatment. The methods disclosed increase the likelihood that a clinician can successfully treat cancer and immune related disease extremely quickly. Compared with traditional immune drug efficacy screening, the present invention can significantly shorten the duration of time for immune drug efficacy screening, as well as increase the survivability of disease for clinical patients. The current methods provide the best treatment outcomes for patients while also being fast enough that it is possible to go from diagnosis to a treatment plan within a matter of days.

In the current art, drug sensitivity screening of existing chemotherapeutic drugs or tumor-targeted drugs examines only the tumor cells, which do not contain immune cells because most immune cells are removed during processing. No screening method for drugs targeting immune cells or immune system regulation currently exists. Through numerous experiments, the present invention discloses a new animal model and device to detect the effects and mechanisms of immune drugs against tumors, as well as screening suitable biomarkers for immune drug reactivity, thereby achieving rapid screening of immune system targeting or optimizing cancer drug treatment.

### FIGURE LEGENDS

FIG. 1 is a schematic diagram of a method for rapid screening of immune drugs by “IO-FIVE (Immuno-Oncology Fast In Vivo Efficacy test)” technology. Tumor immunotherapy is taken as an example. Tumor tissues derived from a patient are taken and processed to make the tumor cells and autologous immune cells mixed in a certain ratio (recommended 1.5:1 to 15:1) and the capsule tubes are sealed, and the immunodeficient mice are inoculated subcutaneously or orthotopically and administered drug. After 5 to 14 days, the capsule tubes are taken out for the detection of total cell viability, the ratio of cellular subgroups, cell phenotypes as identified by flow cytometry (FACS), and various omics (e.g., transcriptome) are analyzed before and after drug administration.

FIG. 2 shows the phenotype detection of single cell suspension (in the capsule tubes) of tumor tissues by flow cytometry on day 0 for a patient with ovarian cancer (see Example 1).

FIG. 3 shows the detection of the viability of total cells (containing tumor cells and immune cells) in the capsule tubes on day 10 as measured by chemiluminescence (CTG method) for the patient with ovarian cancer (see Example 1).

FIG. 4 shows the phenotype analysis of the single cell suspension in the capsule tubes on day 10 by flow cytometry (FACS) for a patient with ovarian cancer (see Example 1).

FIG. 5 shows the phenotype detection of the single cell suspension of tumor tissues (in the capsule tubes) by flow cytometry on day 0 for a patient with ovarian cancer (see Example 2).

FIG. 6 shows the detection of the viability of total cells containing tumor cells and immune cells in the capsule tubes on day 10 by CTG cell viability chemiluminescence method for the patient with ovarian cancer (see Example 2).

FIG. 7 shows the phenotype detection of tumor tissues (tumor cells and immune cells) in the capsule tubes on day 10 by FACS for a patient with ovarian cancer (see Example 2).

FIG. 8 shows the phenotype detection of different types of single cell suspension of metastatic tumor tissues (tumor tissues before purification, tumor cells after purification, immune cells after purification, tumor samples after addition of immune cells) on day 0 by flow cytometry for a patient with ovarian cancer (see Example 3).

FIG. 9 shows the detection of the viability of total cells containing tumor cells and immune cells in the capsule tubes on day 10 by CTG cell viability method for a patient with ovarian cancer (tumor tissues before purification, and tumor samples after addition of immune cells), (see Example 3).

FIG. 10 shows the phenotype detection of tumor tissues (tumor cells and immune cells) in each of the capsule tubes by FACS on day 10 for the patient with ovarian cancer (see Example 3).

FIG. 11 shows the phenotype detection of single cell suspension of tumor tissues (in capsule tubes) on day 0 by flow cytometry for a patient with lung cancer (see Example 4).

FIG. 12 shows the detection of the viability of total cells (containing tumor cells and immune cells) in the capsule tubes on day 14 by the CTG cell viability method for the patient with lung cancer (see Example 4).

FIG. 13 shows the phenotype detection of tumor tissues (tumor cells and immune cells) in the capsule tubes on day 14 by FACS for the patient with lung cancer (see Example 4).

FIG. 14 shows the phenotype detection of single cell suspension (in the capsule tubes) of tumor tissues (peripheral blood tumor) on day 0 by flow cytometry for two acute myeloid leukemia (AML) patients (see Example 5).

FIG. 15 shows the detection of the viability of total cells (containing tumor cells and immune cells) in the capsule tubes on day 14 by CTG cell viability method for two acute myeloid leukemia (AML) patients (see Example 5).

FIG. 16 shows, A. the phenotype detection of tissues (tumor cells and immune cells) in the capsule tubes by FACS on day 14 for an acute myeloid leukemia (AML) patient of immunoreactive type #124, and B. the analysis of the ratio of CD8+T in live cells on day 14 by FACS for two AML patients, #124 (immune responder type) and #123 (immune non-responder type), (see Example 5).

FIG. 17 shows, A. the heat-map showing transcriptomic differential gene signaling pathways of the two AML patients on day 0, and, B. the comparative analysis of tumor proliferation level, angiogenesis level, neutrophil activity level and effector (T) cell level biomarkers for two AML patients with differential gene expression of clusters (see Example 5).

FIG. 18 shows the analysis of the differential expression of key membrane protein molecules in genes for two acute myeloid leukemia (AML) patients (see Example 5).

FIG. 19 shows genomic DNA analysis including genomic CNV and mutation for two acute myeloid leukemia (AML) patients (see Example 5).

FIG. 20 shows the phenotype detection of single cell suspension (in the capsule tubes) of tumor tissues (peripheral blood and bone marrow in combination with peripheral blood) on day 0 by flow cytometry for three acute myeloid leukemia (AML) patients (see Example 6).

FIG. 21 shows the detection of the viability of total cells (containing tumor cells and immune cells) in the capsule tubes on day 14 by CTG cell viability method for three acute myeloid leukemia (AML)patients (see Example 6).

FIG. 22 shows the phenotype detection of tumor tissues (tumor cells and immune cells) in the capsule tubes by FACS on day 14 for three patients (see Example 6).

FIG. 23 shows the phenotype detection of single cell suspension of tumor tissues (bone marrow in combination with peripheral blood) on day 0 by flow cytometry for an acute myeloid leukemia (AML) patient (see Example 7).

FIG. 24 shows the detection of the viability of total cells (containing tumor cells and immune cells) in the capsule tubes on day 14 by CTG cell viability method for an acute myeloid leukemia (AML)patient (see Example 7).

FIG. 25 shows the detection of the ratio of tumor tissues (cancer cells and immune T cells) in the capsule tubes on day 14 by FACS for the patient (see Example 7).

FIG. 26 shows the phenotype detection of single cell suspension (in capsule tubes) of tumor tissues (bone marrow) on day 0 by flow cytometry for two acute myeloid leukemia (AML) patients (see Example 8).

FIG. 27 shows the detection of the viability of total cells (containing tumor cells and immune cells) in the capsule tubes on day 10 by the CTG cell viability chemiluminescence method for two acute myeloid leukemia (AML) patients that includes two experiments: (1) the experiment where the same mouse is inoculated with the capsule tubes containing the cells from the two patients (two patients in one mouse) and (2) an experiment where one mouse is inoculated with the capsule tubes containing the cells from one patient (one patient in one mouse), (see Example 8).

FIG. 28 shows the detection of the ratio of tumor tissues (tumor cells and immune cells) in the capsule tubes on day 10 by FACS for these two acute myeloid leukemia (AML) patients (see Example 8).

### DETAILED DESCRIPTION

The present invention solves the above-mentioned problems through the following technical solutions:

Treating a cancer patient with an immunomodulatory drug comprising the steps of:
a) isolating tumor cells and immune cells from the patient in a single cell suspension,
b) combining the tumor cells with autologous patient lymphocytes in an implantable capsule,
c) implanting the capsule in an immunodeficient mouse for a time,
d) administering an immunomodulatory drug to the mouse during this time,
e) removing the capsule from the mouse and analyzing the total cell viability and cell surface markers, and
f) administering the immunomodulatory drug to the patient upon the condition that the immunomodulatory drug decreased the cell viability of the tumor in the capsule or increased the ratio of lymphocytes in the capsule.

An immunomodulatory drug is a drug that is to be used in immunotherapy, which is defined as any treatment wherein the target mechanism of action of the drug is via an immune cell. In all embodiments, the term “immunomodulatory drug” means that the drug targets an immune cell involved in regulating an anti-cancer response or targets an immune cell effecting an anti-cancer response, or some combination thereof. When an immunomodulatory drug is used to treat cancer in the clinic, it is termed an “immunotherapy drug” since its immunomodulatory effect is expected to be clinically therapeutic. Immunotherapy drugs include but not limited to checkpoint inhibitor therapy for cancer such as PD1 antagonists (anti PD1 or anti PD-1L) and CTLA4 antagonists (anti CTLA4). Some cancer cells make high levels of proteins that switch off T cells when they should instead be attacking the cancer cells. Such T cells can no longer recognize and kill cancer. Drugs that block checkpoint proteins are termed checkpoint inhibitors. Checkpoint inhibitors reactivate the immune system so that T cells are able to find and attack the cancer cells. Checkpoint inhibitors that block PD-1 include nivolumab (Opdivo) and pembrolizumab (Keytruda). Ipilimumab (Yervoy) is a checkpoint inhibitor drug that blocks CTLA-4. Checkpoint inhibitors that block PD-Ll include: atezolizumab, avelumab, durvalumab. Testing drugs that affect HVEM, also known as CD270, are also contemplated because CD270 has been shown to be an immune cell checkpoint receptor belonging to the tumor necrosis factor receptor (TNFR) superfamily. Notably, checkpoint inhibitors are not the only way to activate or derepress T cells. Other immunotherapies for cancer include CART-T cells, cytokine therapy, therapeutic vaccine, mitogens, etc. The methods disclosed herein are not limited to any particular immunotherapy.

Typical immune cells to be used in the methods described herein are T cells (e.g., CD3+ α/β cells or γ/δ cells including phenotypically CD45^{high} SSC-A^{low} CD3+T cells), including CD8+ cytotoxic T lymphocytes (CTL), CD4+ helper lymphocytes (e.g., Th1, Th2, Th17, Tfh, Th9 and Th22, etc.), and T regulatory cells (e.g., Foxp3+ Tregs, CD4+CD25+ Treg, CD8+ Treg). All the T cells in the tumor microenvironment or systemic circulatory pool can be expressed immune checkpoints (e.g., PD1, CTLA4, Lag3, Tigit, Tim3, VISTA, etc.), T cell (co)activation components (e.g., CD28, 4-1BB/CD137, OX40/CD134, GITR, etc.), and chemokine receptors (CCR2, -4, -5, -6, -7 and -8, CXCR3, -4 and -5, etc.). T cell progenitors and stem cells may also be used. Additional types of lymphocytes can also be used according to the methods, including Natural killer cells (NK), B cells, macrophages, dendritic cells, mast cells, neutrophils and other immune cells. In certain embodiments, the lymphocytes may comprise antigen presenting cells (e.g., B cells, dendritic cells and macrophages). Cells of non-lymphoid origin may also be added to the lymphocytes.

For the mixture of cells in the capsule, the ratio of the patient's tumor cells to T cells can be a critical parameter for useful screening of an immunomodulatory drug in the described methods. If there are too many tumor cells in the mixture, then the T cells response will be overwhelmed, e.g., excessive immune-suppression signals, such as ADAM10/17 mediated soluble PDL1 section, high level of Tim3, lag3, tigit and other checkpoints expression, presented by the tumor tissue blunts the anti-PD1 response in that repeated and chronic tumor antigen stimulation induces T cell exhaustion. Alternatively, if there are too many T cells in the mixture, then the proliferation of the T cells will be muted for lack of proper antigenic stimulation, as well as covering the actual tumor cell growth capability due to the lymphocytes overactivation. Thus, the ratio of cancer cells to T cells (the CT ratio) in testing checkpoint inhibitors should be at least 1:1, but more preferably 1.5:1, 2:1, 3:1; 4:1, 5:1. 6:1, 7:1, 8:1, 9:1, 10:1 up to 15:1.

For PD-1/PD1L and CTLA4 screening, a critical range of tumor cells to T cells is required, specifically 1.5:1 to 15:1 C/T ratio. Such a range is not determinable using routine titration efforts since each cell growth pattern and resistance to drug is unique to each of the patients. The discovery of this critical range was not routine or predictable because of the nature of individual human variability of tumors and T cells both genetically due to mutation and TCR rearrangement and epigenetically due to silencing of particular alleles.

Almost all tumors naturally comprised autologous T cells, more in hot tumor while less in cold tumor, in solid cancer as well as blood cancer. “Cold tumors” are characterized by the lack of T-cell infiltration, whereas “hot tumors” are not. Consequently, the T cells in the implanted capsule may comprise tumor infiltrating lymphocytes (TIL), peripheral blood mononuclear cells, bone marrow cells, or some combination thereof and other cells isolated in conjunction with the tumor or using frozen samples. Tumor-infiltrating lymphocytes include T cells and B cells. They are one of the key parts of the larger category of ‘tumor-infiltrating immune cells’ which include both mononuclear and polymorphonuclear immune cells. Traditional digestion and processing of tumors by flow cytometry or magnetic bead separation deplete the lymphocytic component (tumor infiltrating lymphocytes) from these processed tumors. Thus, traditional techniques for creating cell suspensions for patient derived xenografts (“PDX”) cannot be used for screening immunomodulatory drugs since the target T cells are not present in the processed xenograft.

It Is contemplated that the mixture of T cells and tumor cells within a capsule produce a detectable signal for the immunotherapeutic effect. For embodiments testing cancer immunotherapy in a single mouse, the signal is measured from the proliferation of tumor cells in capsules that either do not have autologous T cells included or, if T are included, then the capsules' molecular weight cutoff excludes a therapeutic agent which exceeds the molecular weight permeability of the polymeric membrane. Antibodies are typical of a large molecular weight drug, usually around 160 kDa, below the limitation of the average pore permeability of the polymeric membrane here (300-1000 kDa). Capsules can be implanted in untreated control mice, while other capsules can be implanted in an experimental treatment mouse that received the drug. In either event, the signal can be measured by impairment of proliferation of tumor tissue cells in the capsule relative to the untreated group.

Any method in the art for detection of tumor cell proliferation may be used. However, in one embodiment, it is more convenient to use a marker for tumor cell proliferation, such as metabolic ATP production. A convenient method to measure cell viability is to use luminescence producing ATP production reactants, including oxyluciferin, available commercially as Cell Titer Glo™ (“CTG” cell viability detection method). Other metabolic reactants, such as radiolabeled molecules and colorimetric reagents, can also be used. Absolute and relative cell counts can be determined by using a microscope, flow cytometry, etc. The ratio of cancer cells (CD45^{low/-}, CD33⁺, CD38⁺ cancer cells) to T cells (CD3+ T cells) for the mixture is referred to in the examples and drawings as “C/T”, cancer to T cell ratio. Those of skill in the art recognize that the ratio can be expressed as an integer, a percentage, and even as a ratio in reverse order, depending on the context without affecting the meaning of the disclosed ratio.

In certain embodiments, the optimal choice of therapy for a cancer patient is to be analyzed by the amount of metabolic activity of the cells in the capsule after implantation and drug treatment. The term “optimal” is used in a clinically specific manner. The drug (or combination) must be (a) capable of arresting the growth of the tumor in the capsule and (b) arrest the growth of the tumor to a degree that is comparatively better than no treatment as well as being better than any other of the co-tested drugs. A closely related term would be “efficacious;” however, when more than one drug tests positive as being efficacious, discernment of the “optimal” treatment choice is still required. Wherein two or more drugs perform better than the vehicle control (i.e., no treatment), the drugs can be combined for clinical treatment; however, the term “optimal” still means the drug that is the most effective in stopping or reducing the growth of the implanted tumor of the available choices. When one drug performs co-equivalently to a different drug in the readout, both drugs are to be considered “optimal” for treatment, and the clinician should look to secondary factors for choosing treatment, such as the likelihood of side effects and relative cost.

While the decision to combine any “optimal” therapeutic drug with another for treatment may be based on the combinations ability to be mutually synergistic or mutually additive in arresting cancer growth in the capsule, in no instance should a mutually antagonistic combination be used for therapy. In other words, if two drugs inhibit the effects of each other on tumor growth, the clinician should not combine the drugs in treating a patient for cancer. Thus, one of the purposes of the disclosed methods is to exclude combination therapies that are antagonistic to each other as well as define the optimal treatment.

Based on the readout from the methods, a patient can first be screened for being a “responder” or “non-responder” to any particular drug when considering monotherapy. To be deemed a responder, patients' cell tumor viability after implantation and treatment in the animal should be analyzed with the Students' t-test, wherein the p value should be below 0.05 (p<0.05) between treatment and vehicle, and the mean value of luminescence in treatment versus vehicle groups should be less than 50%. In alternative embodiments, if the p value for the difference in luminescence between vehicle controls and experimental groups is not significant (p>=0.05), a patient can still be a “responder” to the drug where the mean luminescence value is less than 50% or where T lymphocytes have been significantly activated or boosted. Such patients are deemed “immune responders” and demonstrate an immune response to the drug but with a relatively low effector response in killing tumor cells. When testing only one drug, a positive signal indicating that a patient will successfully respond to treatment is determined by comparing the experimental group to the control group. Any decrease in proliferation relative to controls is deemed a positive response to the drug, as long as the difference in signal between control and experimental is statistically significate as defined herein. When testing multiple drugs in combination, whether in the same mouse or not, the statistical significance is first compared to control to determine a threshold response. The drugs are then ranked as to effectiveness. Statistically, the various drug treatments should be ranked against each other, wherein the p value of one drug versus the other is significant (p<0.05) using the Mann-Whitney test.

The efficacy of drugs can be determined concomitantly with the growth state, extent of apoptosis, and the degree of differentiation of the tumor cell along with measuring the T cell proliferation, activation or differentiation. Such parameters are usually measured by flow cytometry (FACS). When the cells from the post-treatment capsule are analyzed by FACS, the percentage of T lymphocytes can be increased, or the lymphocytes' activation can be enhanced after drug treatment compared with vehicle, as observed by a change in cell markers. This flow cytometry data can further assist in determining if a patient should be classified as a “responder;” however, such data is not essential to determine this status.

In summary, a “responder” has the following criteria:
(1) Tumor cell proliferation (CTG method): t-test p value should be below 0.05 (p<0.05) between treatment and vehicle, and luminescence mean value of treatment vs. vehicle should be less than 50%.
(2) FACS data (not essential): The percentage of T lymphocytes should be increased or its activity enhanced after immune drugs treatment compared with vehicle.
(3) Alternatively,
1) if the p value for proliferation (CTG method) is not significantly different than vehicle control (p>=0.05), but the total luminescence mean value is less than 50% and/or T lymphocytes have been significantly activated or boosted, then the patient will likely respond, or at least partially respond to the drug.
2) if the tumor cell proliferation (CTG method) mean value is no different with vehicle group but FACS data showed T cells overwhelm the tumor cells and are activated, then then the patient will likely respond, or at least partially respond to the drug.

In contrast, a “non-responder” has the following criteria:
(1) Tumor cell proliferation (CTG method): t-test p value is greater than 0.05 (p>0.05) between treatment and vehicle, and luminescence mean value of treatment is more than 70% of the vehicle control.
2) FACS data (not essential): there is no phenotyping difference in treatment groups compared with vehicle.

In the event a patient does not respond to the drug without concomitant tumor cell killing, then the drug would not be the optimal treatment. However, the drug should still be considered part of the clinician's arsenal to treat the patient, either as second line therapy or to be combined with another drug, such as a chemotherapeutic agent.

In the embodiments of the invention once a clinically relevant drug or combination thereof is identified, the dose can be titrated in the mouse model to determine the efficacy of the dose. As such, the optimal dose will be that which induces the optimal responder phenotype. For example, as the dose of a drug identified as effective is increased, the tumor killing will increase, but only to a maximum of 100% killing of the tumor cells. If a maximal dose response (or plateau in response) is identified, then the clinician will be informed so that the drug dose in a patient can be managed clinically with the least amount of toxicity. Generation of such dose response profiles are specifically contemplated as part of the invention; however, a dose response profile is not necessary to implement the teachings herein.

Immunomodulatory drugs used in immunotherapy include but not limited to immune cell checkpoint inhibitors targeting for PDL1/PD1, CTLA4, Lag3, Tim3 and Tigit, immune agonists targeting 4-1BB, CD28, OX40, as well as those bispecific antibodies or T cell engagers with one arm for tumor antigen binding and another for T cell activation, such as HLA-A2-gp100/anti-CD3 bispecific fusion protein (Tebentafusp), BCMA/CD3 bispecific antibody (Teclistamab); or bispecific antibodies targeting for two epitopes of the immune molecules, such as PD-1/CTLA-4 bispecific antibody (Cadonilimab), as well as bispecific or tri-specific antibodies targeting multiple tumor associated antigens and immune cells, such as CD19xBCMAxCD3 antibody, BCMAxGPRC5DxCD3 antibody (MBS314). In certain embodiments, immunomodulatory drugs used in immunotherapy include drugs targeting the innate immune system, such as NK cell engagers (e.g., CD30/CD16 bispecific antibody) or ADCC (antibody dependent cell cytotoxicity, such as CD38 antibody Daratumumab) drugs to unleash NK cytotoxicity against tumor cells, and enhanced ADCP (antibody dependent cell phagocytosis) drugs or macrophage checkpoint blockers by targeting CD47/Sirpa, CD24/Siglec10 pathways. Basically, IgG1 structure-like antibodies or fusion proteins with high affinity for all Fc receptors are potent activators of ADCC and ADCP.

Targeted therapy is one type of cancer treatment. The drugs targeting specific genes and proteins essential for cancer development and metastasis, e.g., TrastuzumAb (Her2), Osimertinib (EGFR T790M), niraparib (PARP1/2), chidamide (HDAC1/2/3/8/10/11), venetoclax (Bc12), etc. Targeted therapy can also target the tumor microenvironment, such as blood vessel cells, e.g., Bevacizumab (VEGF). In certain embodiments, antibody-conjugated drug is one type of targeted therapy, which combines monoclonal antibodies specific to surface antigens present on particular cancer cells with highly potent anti-cancer agents linked via a chemical linker, e.g., DS8201 (Trastuzumab deruxtecan). Molecular glue and PROTAC (proteolysis targeting chimera) are also considered as different types of targeted therapy, e.g., Lenalidomide (IKZF1/3).

The term “chemotherapeutic agent” is to be interpreted as an agent that has a direct mechanism of action on tumor cells or/and tumor microenvironment. Chemotherapeutics can be small molecule chemicals, or large molecule biologics (steroid/corticosteroid medicine, e.g., prednisone/dexamethasone, and antibody-conjugated drugs, e.g., DS8201). Specific examples of chemotherapeutic drugs compatible with the disclosed methods include but are not limited to paclitaxel, pegaspargase, gemcitabine, Oxaliplatin/Cisplatin, azacitadine (5AZA), Bendamustine, cyclophosphamide, doxorubicin hydrochloride (hydroxydaunorubicin), vincristine sulfate (Oncovin), bortezomib, lenalidomide, mitoxantrone and irinotecan. In certain embodiments, general chemotherapy includes cancer targeted therapeutic drugs without targeting immune system, e.g., TrastuzumAb (Her2), Osimertinib (EGFR T790M), niraparib (PARP1/2), chidamide (HDAC1/2/3/8/10/11), venetoclax (Bc12), Bevacizumab (VEGF), rituximab(CD20), Ibrutinib(BTK), Alpelsib(PIK3CA), anlotinib(multi-kinase inhibitor), etc.

In some embodiments, the primary tumor cell is obtained from an ex vivo tumor tissue sample. The ex vivo tumor sample can be obtained from a patient, or a PDX model established by using a tumor cell of the patient. In embodiments, the animal is a mouse, preferably a nude mouse, SCID mouse, a BALB/c Nude mouse, an NCG mouse, a NSG mouse, and a NOD-SCID mouse or other immunocompromised mouse incapable of mounting an immune response to the xenograft. In other embodiments, the sensitivity of the tumor cell to any particular candidate drug can be determined in vitro. In vivo and in vitro dosages and administration schedules are defined by known mass/weight/time relationships for the effective use of the treatment drug, although titrations can be used for optimizing a dose-response curve. In embodiments, the candidate drug is administered to an animal orally or parenterally. In embodiments, the primary tumor cell is an isolated tumor cell that has been digested and sorted. In embodiments, the sensitivity of the tumor cell to the candidate drug is determined 5-14 days after administering the candidate drug to the animal, preferably 7-10 days after administering the candidate drug to the animal. The timing of the subsequently treatment from removing the capsule is rapid. For use in the clinic in one embodiment, the physician would select the optimal immunomodulatory agent and begin clinical treatment within a time of two days, three days, four days, five days, six days, seven days, eight days, nine days, ten, eleven, twelve, thirteen, or fourteen days from removing the capsule and the readout of the results obtained by the methods disclosed herein.

In embodiments, the implant medical device is a tubular capsule having a molecular weight cut-off value of about 500,000 Dalton, preferably a modified polyvinylidene fluoride (PVDF) tube having a molecular weight cut-off value of about 500,000 Dalton, more preferably a modified PVDF tube with an inner diameter of about 1-2 mm having a molecular weight cut-off value of about 500,000 Dalton. In some embodiments of the present invention, the average protein permeability of the capsule tube is 300-1,000 Kda, preferably 500-700 kDa. In others, the average pore size of the polymer was about 300 kDa-700 kDa. Selection of a specific pore size is to allow the ingress and egress of macromolecular proteins but not allowing free ingress and egress of cells. The terms “tube,” “capsule,” “capsule tube,” “mini-capsule” and “device” are to be read as synonymous unless a predicate description indicates otherwise. In some embodiments, the kit also includes an insertion page, which includes the user manual for the kit.

A primary tumor cell refers to a tumor cell obtained from an ex vivo tumor tissue sample. In embodiments, the primary tumor cell is isolated from a tumor tissue sample of a patient, including but not limited to a tumor tissue that is clinically removed, a tumor biopsy sample, etc. In embodiments, the primary tumor cell is obtained from a PDX mouse model that comprises a tumor cell from a tumor tissue of a patient.

The tumor cells can derive from tissue of various types of tumors, including but not limited to tumors located in the following parts of the body: the digestive tract (such as the stomach, intestine, duodenum, colon, pancreas, bile duct, anal canal, etc.), mammary glands, lung, liver, endocrine glands (such as adrenal gland, parathyroid gland, pituitary, testis, ovary, thymus, thyroid gland, etc.), urinary and reproductive system (such as kidney, bladder, ovary, testis, prostate, etc.), skeletal muscle system (such as bone, smooth muscle, striated muscle, etc.), nervous system (such as brain), skin, head and neck, blood system and so on. For example, the primary tumor cell can be derived from a gastric cancer tissue, a duodenal cancer tissue, or a lung cancer tissue. The tumor cells can be derived from any type of tumor located in any part of the body.

The methods disclosed herein can be used to treat any cancer type including, but not limited to: acute lymphoblastic leukemia (ALL), acute myeloid leukemia (AML), adrenocortical carcinoma, aids-related cancers, AIDS-related lymphoma, primary CNS lymphoma, anal cancer, appendix cancer, gastrointestinal neuroendocrine tumors, astrocytomas (brain cancer), atypical teratoid/rhabdoid tumor, central nervous system (brain cancer), basal cell carcinoma of the skin, bile duct cancer, bladder cancer, bone cancer (including Ewing sarcoma and osteosarcoma and malignant fibrous histiocytoma), brain tumors, breast cancer, bronchial tumors (lung cancer), Burkitt lymphoma, carcinoma of unknown primary, central nervous system tumors, atypical teratoid/rhabdoid tumor (brain cancer), medulloblastoma and other CNS embryonal tumors (brain cancer), germ cell tumor (brain cancer), primary CNS lymphoma, cervical cancer, childhood cardiac tumors treatment, cholangiocarcinoma—see bile duct cancer, chordoma (bone cancer), chronic lymphocytic leukemia (CLL), chronic myelogenous leukemia (CML), chronic myeloproliferative neoplasms, colorectal cancer, craniopharyngioma (brain cancer), cutaneous t-cell lymphoma (mycosis fungoides and Sézary syndrome), ductal carcinoma in situ (DCIS), embryonal tumors, medulloblastoma and other central nervous system (brain cancer), endometrial cancer (uterine cancer), ependymoma (brain cancer), esophageal cancer, esthesioneuroblastoma (head and neck cancer), extracranial germ cell tumor, extragonadal germ cell tumor, eye cancer, intraocular melanoma, retinoblastoma, fallopian tube cancer, gallbladder cancer, gastric (stomach) cancer, gastrointestinal neuroendocrine tumors, gastrointestinal stromal tumors (GIST) (soft tissue sarcoma), germ cell tumors, childhood central nervous system germ cell tumors (brain cancer), childhood extracranial germ cell tumors, extragonadal germ cell tumors, ovarian germ cell tumors, testicular cancer, gestational trophoblastic disease, hairy cell leukemia, head and neck cancer, heart tumors, hepatocellular (liver) cancer, langerhans cell histiocytosis, Hodgkin lymphoma, hypopharyngeal cancer (head and neck cancer), intraocular melanoma, islet cell tumors, pancreatic neuroendocrine tumors, kaposi sarcoma (soft tissue sarcoma), kidney (renal cell) cancer, langerhans cell histiocytosis, laryngeal cancer (head and neck cancer), leukemia, lip and oral cavity cancer (head and neck cancer), liver cancer, lung cancer (non-small cell, small cell, pleuropulmonary blastoma, pulmonary inflammatory myofibroblastic tumor, and tracheobronchial tumor), lymphoma, male breast cancer, melanoma, intraocular (eye) tumors, merkel cell carcinoma (skin cancer), mesothelioma, metastatic cancer, metastatic squamous neck cancer with occult primary (head and neck cancer), midline tract carcinoma with NUT gene changes, mouth cancer (head and neck cancer), multiple endocrine neoplasia syndromes, multiple myeloma/plasma cell neoplasms, mycosis fungoides (lymphoma), myelodysplastic syndromes, myelodysplastic/myeloproliferative neoplasms, myelogenous leukemia, chronic CML, myeloid leukemia, myeloproliferative neoplasms, chronic, nasal cavity and paranasal sinus cancer (head and neck cancer), nasopharyngeal cancer (head and neck cancer), neuroblastoma, neuroendocrine tumors (gastrointestinal), non-Hodgkin lymphoma, non-small cell lung cancer, oral cancer, lip and oral cavity cancer and oropharyngeal cancer (head and neck cancer), osteosarcoma and undifferentiated pleomorphic sarcoma of bone, ovarian cancer, pancreatic cancer, pancreatic neuroendocrine tumors (islet cell tumors), papillomatosis (childhood laryngeal), paraganglioma, paranasal sinus and nasal cavity cancer (head and neck cancer), parathyroid cancer, penile cancer, pharyngeal cancer (head and neck cancer), pheochromocytoma, pituitary tumor, plasma cell neoplasm/multiple myeloma, pleuropulmonary blastoma (lung cancer), pregnancy and breast cancer, primary central nervous system (CNS) lymphoma, primary peritoneal cancer, prostate cancer, pulmonary inflammatory myofibroblastic tumor (lung cancer), rectal cancer, recurrent cancer, renal cell (kidney) cancer, retinoblastoma, rhabdomyosarcoma (soft tissue sarcoma), salivary gland cancer (head and neck cancer), sarcoma, childhood rhabdomyosarcoma (soft tissue sarcoma), childhood vascular tumors (soft tissue sarcoma), Kaposi sarcoma (soft tissue sarcoma), osteosarcoma (bone cancer), soft tissue sarcoma, uterine sarcoma, skin cancer, small cell lung cancer, small intestine cancer, soft tissue sarcoma, squamous cell carcinoma of the skin squamous neck cancer with occult primary, metastatic (head and neck cancer), stomach (gastric) cancer, t-cell lymphoma, testicular cancer, throat cancer (head and neck cancer), nasopharyngeal cancer, oropharyngeal cancer, hypopharyngeal cancer, thymoma and thymic carcinoma, thyroid cancer, tracheobronchial tumors (lung cancer), transitional cell cancer of the renal pelvis and ureter (kidney renal cell cancer), carcinoma of unknown primary origin, ureter and renal pelvis cancer, transitional cell cancer (kidney renal cell cancer), urethral cancer, uterine cancer, endometrial cancer, uterine sarcoma, vaginal cancer, vascular tumors (soft tissue sarcoma), vulvar cancer, Wilms tumor and other childhood kidney tumors.

In embodiments, the primary tumor cell is from a tumor cell that has been digested and sorted. In embodiments, digestion is carried out as follows: remove the non-tumor tissue and necrotic tissue, cut the tumor samples into small cubes, rinse with HBSS, collect the pellets and use 1× collagenase at 37° C. to digest for 1-2 hours. In embodiments, sorting is carried out as follows: dilute with serum medium (1:1) to terminate digestion and run through 70 μm screen mesh; collect cell suspension and centrifuge the suspension at 1000 rpm for 3 minutes to remove supernatant, and re-suspended in PBS containing 1% FBS; adjust cell density to 1×10⁸/ml; add CD45 cells sorting magnetic beads or/and fibroblasts sorting magnetic beads at a concentration of 20 μl/10⁷ cells, incubate for 30 min at room temperature. Cells are rinsed with PBS containing 1% FBS and re-suspended with 2 ml PBS containing 1% FBS. The magnetic beads are mounted on the magnet column and washed with PBS containing 1% FBS; then the re-suspended cells are loaded onto the magnetic column. Collect the liquid draining off the column(tumor cells). The column is then removed from the rack, rinsed with 5 mlPBS(1% FBS) by using the syringe, and the outflow is collected (CD45+ immune cells). All the collected liquid is centrifuged at 400 G for 5 minutes to remove the supernatant. The cells are resuspended in the culture medium, counted, and adjusted to a concentration of 1−10×10⁵/ml.

The candidate drug in the present invention may be a known immunomodulatory anti-tumor drug or a combination of known anti-tumor drugs, a new anti-tumor drug or combination of new anti-tumor drugs, or a new combination of known anti-tumor drugs. In the methods of the invention, the drugs to be measured may be used in the form of solid, semisolid, or liquid. The drug may be administered to the mouse at a desirable frequency as required.

In the method of the present invention, the candidate drug can be administered to the animal orally or parenterally (such as via intravenous, intramuscular, subcutaneous or intravenous infusion), topical administration, inhalation, and transdermal delivery such as skin patches, implants, suppositories, etc. A skilled person in the art will choose a suitable route of administration according to needs.

In embodiments of the present invention, the effect of the candidate drug can be determined 2-14 days after administering the drug to the animal, preferably 7-10 days after administering the drug to the animal. In the present invention, the endpoint signal is metabolic activity such as growth and proliferation, as measured by the following methods including but not limited to ATP detection, CCK8(Cell Counting Kit-8), MTT, BrdU labeling, Ki67 labeling and so on. In addition, the effect of the drug can be measured real-time in vivo through fluorescent tomography and even through serial withdrawal of the implanted capsule contents using a fine needle syringe.

In embodiments of the invention, the implantation device can be implanted subcutaneously into the animals. A skilled person in the art will understand that a desired implantation method known in this field can be selected according to the need thereof.

The methods of the present invention also enable primary tumor cells, especially isolated or sorted tumor cells, to be grown with autologous lymphocytes in the implanted devices in the experimental animals (such as mice). The animal thus acts as a host to enable the primary tumor cell to grow in the in vivo environment. The disclosed methods can be combined with in vivo and in vitro experimental techniques to carry out rapid and efficient antitumor drug evaluation. The disclosed methods have the advantages of time-efficiency, convenient operation, low cost, repeatability, and applicability to the rapid and accurate detection the effects of an immunomodulatory drug.

The present invention further provides a polymeric capsule in which primary patient tumor cancer cells and immune cells are combined into a single cell suspension. The disclosed devices can comprise tumor cells and immune cells that are derived from fresh tumor tissues or body fluids of clinical patients, meaning that the sample is collected, delivered to the laboratory within 72 hours (ideally 24 hours) before the sample is being processed and implanted into the mouse.

In some embodiments of the present invention, the tumor tissues comprise surgically resected tumor tissues or biopsied tumor tissues from surgery.

In some embodiments of the present invention, the body fluids comprise one or more of blood, bone marrow, pleural fluid, ascites, and cerebrospinal fluid.

In some embodiments of the present invention, the capsule tube needs to be pre-processed by activating, flushing with ultrapure water, and autoclaving before use, wherein an activating mode is preferably dehydrated alcohol.

In some embodiments of the present invention, the tumor cells are those that do not bind with the column and flow through; the immune cells are autologous immune cells enriched by binding with the column and sorting into a single cell suspension, wherein they are derived from tumor-infiltrating lymphocytes in solid tumor tissue of a patient, or the peripheral blood mononuclear cells in peripheral blood of a patient.

In some specific embodiments of the present invention, the sorting equipment used in said sorting comprises:
(1) sorting magnetic beads, preferably anti-human CD45 microbeads, or anti-human CD45 microbeads and anti-human fibroblast microbeads;
(2) and the sorting device is preferably a LS column.

In some specific embodiments of the present invention, the cell suspension is prepared before sorting by the following steps:
(1) removing non-tumor tissue and necrotic tissue from the tumor sample, cutting the tumor sample into tissue blocks with a size of 1-2 mm, washing and centrifuging with HBSS buffer solution containing 1% BPS, and collecting the tumor tissue blocks;
(2) digesting with collagenase, terminating the digestion with a medium containing serum, and collecting the cell suspension; removing the supernatant by centrifugation and then collecting cell pellet;
(3) lysing red blood cells with 3 to 5 times the volume of erythrocyte-lysis solution, removing the supernatant by centrifugation, collecting the cell pellet, and repeating the erythrocyte lysis twice if necessary, wherein the erythrocyte lysis solution comprises 139.6 mmol/L NH₄Cl, 16.96 mmol/L Tris pH 7.2; and
(4) resuspending the cell pellet in PBS with 1% FBS, removing the supernatant by centrifugation and collecting the cell pellet, and adjusting the cell concentration.

In some embodiments of the present invention, the ratio of cancer cells to immune cells ranges from 1.5:1 to 15:1, preferably from 1.5:1 to 9:1.

In some embodiments of the present invention, the percentage of CD8+ T cells in the total live cells is above 0.5%, preferably 0.5%-15%; or wherein the viability of immune cells is more than 20%.

A second aspect of the present invention provides an animal model, which preferably comprises the capsule tube(s) implanted subcutaneously or orthotopically.

In some embodiments of the invention, the animal is a mouse. In some specific embodiments of the present invention, the mouse is an immunodeficient mouse, such as BALB/c nude mouse, NCG mouse, NSG mouse or NOD-SCID mouse.

In some embodiments of the present invention, the number of implanted capsule tube s ranges from 1 to 8.

A third aspect of the present invention provides a method for rapid screening the efficacy of immunoregulating drugs comprising:
(1) administering a drug to be tested to the animal model wherein the drug to be tested is an immunoregulating drug, which comprises one or more of solid, semi-solid and liquid forms; wherein the mode of administration of the drug to be tested comprises one or more of intravenous injection, oral gavage, intraperitoneal injection, and subcutaneous injections.
(2) detecting the total cell viability of tumor and immune mixed cells in the capsule tube, the relative ratio of tumor cells and immune cells, as well as cell phenotype wherein the detecting method of the total cell viability or immune cell viability is preferably chemiluminescence method, e.g., CellTiter Glo; and the detecting method of the relative ratio of different types of cells and cell phenotype is preferably flow cytometry (FACS); and
(3) detecting molecular biological information of tumor cells and immune cells in the capsule tube wherein the detection method of biological information is preferably a DNA sequencing, an RNA sequencing, or combination thereof.

In some specific embodiments of the present invention, steps (2) and (3) are in random order. Preferably, in step (2), the FACS analysis comprises one or more of the following steps:
I. antibody labeling, wherein the antibody comprises one or more of 7AAD, anti-CD45, CD3, CD4, CD8, CD270, CD33, CD38, CD20, PDL1, PD1, CD68, and CD25;
II. performing flow cytometry detection, wherein PBS with 2% FBS is added after said antibody labeling is completed and then loaded for detection in the FACS; and
III. data analysis by software, wherein the software is preferably Flowjo.

A fourth aspect of the present invention provides the use of the capsule tube according to the preparation of an animal model for rapid screening the efficacy of immunoregulating drugs.

In some embodiments of the present invention, the animal model is preferably an immunodeficient mouse.

In some specific embodiments of the present invention, the animal model can perform simultaneous drug screening for 1-8 patients, preferably 1-4 patients.

The above preferred conditions can be combined to obtain the preferred embodiments of the present invention based on the common knowledge in the art.

The reagents and raw materials used in the present invention are all commercially available.

The industrial applicability of the present invention is that it can be used both determine and optimize a treatment regimen for a specific cancer individual in the fastest time possible. The models and methods provided by the present invention provide rapid screening methods for the sensitivity and effectiveness of immune drugs for clinical cancer patients, obtaining pharmacodynamic data from the animal model in vivo, phenotypic identification data of paired flow multicellular subgroups and multi-omics analysis data, and provides rapid and effective functional and mechanistic information for clinical precision medicine and new drug development.

### EXAMPLES

The present invention is further illustrated by way of the following examples, but the invention is not limited to the scope of the described examples. The experimental methods for which specific conditions are not specified in the following examples are chosen according to conventional methods and conditions, or according to the descriptive literature.

### Example 1 Rapid Functional Screening and Potential Biomarker Identification of Immunotherapy for a Patient with Ovarian Cancer

### 1. Experimental Animal Preparation and Sample Collection

BALB/c nude mice (available from Jiangsu Jicui Yaokang Biotechnology Co., Ltd.) aged 6-8 weeks were ordered from the supplier in this experiment and kept in the animal house at SPF level, and the animals were adapted for at least 3 days before the start of the experiment.

Fresh tumor samples from the patient were collected and placed in anticoagulant tubes and transported to the central laboratory by cold chain (2-8° C.) within a short period of time.

### 2. Cell Processing

Non-tumor tissue and necrotic tissue were removed from tumor samples of patients. Tumor samples were cut into tissue pieces of 1-2 mm in size, washed, and centrifuged with HBSS buffer containing 1% PSB (penicillin and streptomycin+Amphotericin B), and tumor tissue pieces were collected. Digestion was performed using 1× collagenase for 1-2 h at 37° C. Said sorting method was as follows:

The digestion was terminated by serum-containing medium (RPMI 1640, Gibco), and the cell suspension was collected with a 70 μM strainer. The supernatant was removed by centrifugation at 1000 rpm for 5 min and the cell pellet was collected. 3-5× lysis solution (139.6 mmol/L NH₄Cl, 16.96 mmol/L Tris, adjusted to pH 7.2 with 1 mol/L HCl) was used to lyse red blood cells at 4° C. for 5 min, the supernatant was removed by centrifuge at 1000 rpm for 5 min. The cell pellet was collected, and lysis of erythrocyte was repeated twice. The cell pellet was resuspended in PBS with 1% FBS, the supernatant was removed by centrifugation at 1000 rpm for 5 min, the cell pellet was collected, and the cell concentration was adjusted to 1×10⁸ cells/ml.

Anti-human CD45 microbeads were added, or anti-human CD45 microbeads and anti-human fibroblast microbeads were added at the concentration of 20 μl/10⁷ cells and incubated at room temperature for 30 min. PBS with 1% FBS was added to rinse the magnetic beads. The supernatant was removed by centrifuge at 1000 rpm for 5 min, and PBS with 1% FBS was used to resuspend the cell pellet. The magnetic beads were loaded on a sorting device (LS column, Miltenyi Biotec). The LS column was washed twice with PBS with 1% FBS. The liquid flowing down from the LS column (tumor cells) and the cells attached to the LS column (enriched immune cells) were collected sequentially. The collected cell suspension was centrifuged at 1000 rpm for 5 min to remove the supernatant. The cells were resuspended in cell culture medium and counted.

In general, tumor tissues need to be purified by adding two both anti-human CD45 microbeads and anti-human fibroblast microbeads so as to preserve the immune cells and stromal fibroblasts from the microenvironment of tumor tissues. However, when the infiltration of immune cells in some tumor tissues was excessive and more than half of the ratio, such as metastatic samples of pleural fluid and ascites of solid tumor, CD45 antibody microbeads were added to enrich or deplete immune cells from the original samples.

### 3. Flow Cytometry Detection and Data Analysis

A single cell suspension of tumor tissue samples of patients before purification, a single cell suspension of tumor cells after purification, and a single cell suspension of enriched immune cells (containing CD3+ T) were collected sequentially. They were then mixed into a suspension of tumor cells and immune cells of different ratios (1.5:1 to 15:1), each containing about 0.1 to 0.3 million cells. For each sample, the suspension was divided into multiple portions according to the purpose of the experiment, and the corresponding flow cytometry antibody mixture was added. The experimental group and control groups were set up using direct antibody labeling and incubated at 4° C. for 25 min in darkness. The preliminary FACS panel for ovarian cancer was labeled with 7AAD and antibodies to CD45, CD3, CD8, CD270, PD1, CD68, and CD25.

After completion of antibody labeling, PBS with 2% FBS was added. The cells were centrifuged at 600 g for 5 min at 4° C. to remove the supernatant. The cell pellet was resuspended by adding 250 μl of 2% FBS (Gibco) in PBS and loaded for flow cytometry detection. Data analysis was performed by Flowjo software.

4. Cells within the Tubing

The capsule device was taken from the biological safety cabinet, i.e., PVDF tube with diameter of 1-2 mm, wherein the average pore size was about 500 KDa molecular weight. The PVDF tube was subjected to anhydrous ethanol activation (or activation with methanol and the like), ultra-pure water flushing, and autoclaving, and the like before being filled with the cell suspension. A capsule tube of 1.5 cm in length was taken and washed repeatedly with cell culture medium (available from Gibco).

Using flow cytometry, the CD45-tumor cells and CD3+ lymphocytes were in a certain ratio (1.5:1 to 15:1). The CD8+ T cells accounted for more than 0.5% of total living cells. The cell suspension was put into PVDF tubes and sealed.

### 5. Inoculation and Drug Administration

The above PVDF tubes were inoculated into mice subcutaneously with a puncture needle. The wounds were sealed with medical tissue glue (available from 3M). The control groups and drug administration groups were chosen, such as Group 1. Paclitaxel, cisplatin and Avastin/Bevacizumab combo-therapy (Bevacizumab, 5 mpk, (mg/kg of body weight), i.p., q.w.*1 (once weekly)+Cisplatin, 2.5 mpk i.v., q.w.*1+Paclitaxel, 10 mpk, i.v., q.w.*1); Group 2. PD1 antibody (10 mpk, i.p., twice per week); Group 3. PD1 antibody combined with the PARP inhibitor Niraparib (PD1 antibody, 10 mpk, i.p., twice per week+Niraparib, 35 mpk, p.o. (by mouth), q.d.*5 (daily for 5 days)); Group 4. Niraparib alone (35 mpk, p.o., q.d.*5), and then the drugs were administered to the groups.

### 6. Cell Viability and Phenotype Detection

3-21 days after administration (day 10 as shown), the mice were euthanized, and the PVDF tubes were removed. Cell viability was quantified using the CellTiter-Glo chemiluminescence method, and the relative ratio and phenotype of tumor cells and immune cells in PVDF tubes were detected by flow cytometry. The changes in the molecular biological information from the tumor cells and immune cells inside of PVDF tubes were detected by molecular biological methods (RNA sequencing and DNA sequencing technology).

### 7. Data Analysis

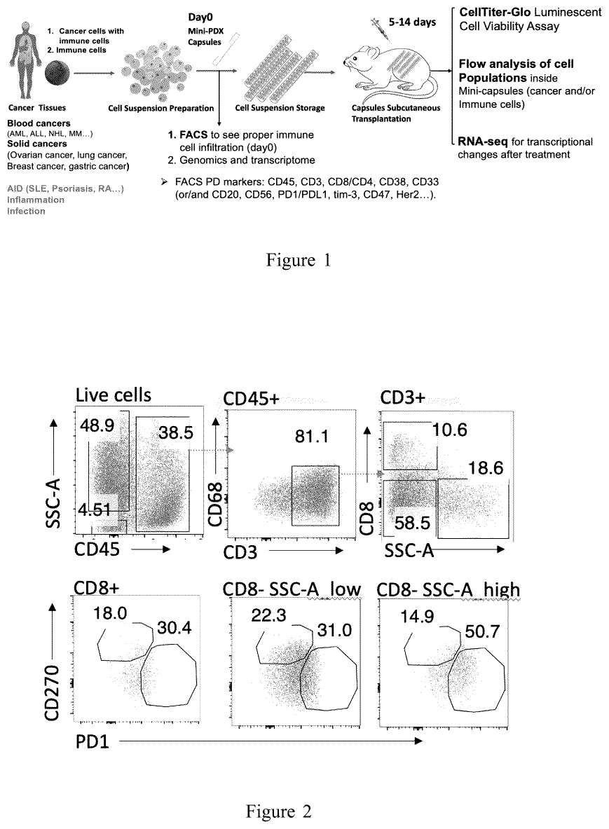

Flow cytometry (FACS) screening on day 0 was as follows. Phenotype analysis of primary tumor sample in capsule tubes indicated that the ratio of CD45-tumor cell and CD3+ T cells equaled 1.6:1, CD8+ T % equaled 3.1%, and tumor infiltrating T cells especially CD8− T cells, highly expressed PD1 molecules, while CD8+ T cells weakly expressed both CD270 and PD1 immune checkpoint molecules, as shown in FIG. 2.

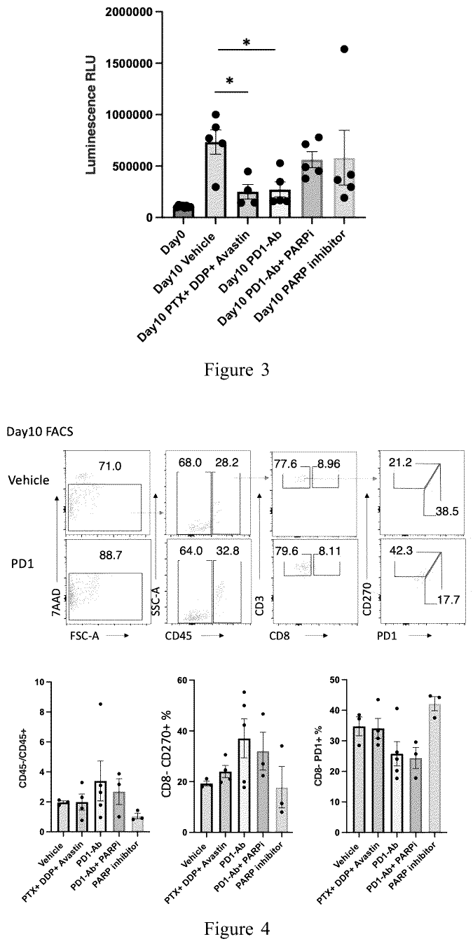

The endpoint of experiments in vivo on day 10 was as follows. Based on the viability of the cells in the control group (as shown in FIG. 3), the proliferation of all cells in the drug administration group was calculated by CellTiter Glo (CTG) assay. The relative ratio and cell phenotype of tumor cells and immune cells in PVDF tubes were detected by flow cytometry (as shown in FIG. 4). The FACS results were analyzed by Flowjo software.

CTG assay and FACS analysis on day 10 showed that PD1 antibody (Ab) could sufficiently renovate tumor infiltrating lymphocytes to kill ovarian cancer cells in PD1 Ab group, nearly the same efficiency as clinical first line chemical combo-therapy (PTX+DDP+Avastin). CD270 molecule (HVEM) was up-regulated accompanied with the down-regulation of PD1 molecules by PD1 Ab therapeutic blocking, either in PD1 Ab mono-therapy group or in PD1 Ab and PARP inhibitor combo-therapy group.

Bioinformatic analysis of tumor cells and immune cells in PVDF capsule tubes of the clinical sample on day 0 or day 10 was carried out by using molecular biological methods (e.g., DNA sequencing, RNA sequencing and other “omics” technologies).

Therefore, the present invention provides a method for the rapid screening of immune drugs by “IO-FIVE” (Immuno-Oncology Fast In Vivo Efficacy test) technology, which is also used in the following examples.

### Example 2 Rapid Functional Screening and Potential Biomarker Identification of Immunotherapy for a Patient with Ovarian Cancer

1. Experimental Animal Preparation, Sample Collection, Cell Processing, Flow Cytometry Detection and Data Analysis Methods, and Cell Sealing Requirements were the Same as in Example 1.

### 2. Inoculation and Drug Administration

As per above PVDF tubes were inoculated into mice subcutaneously with a puncture needle, the wounds were sealed with medical tissue glue (available from 3M), and control groups and drug administration groups were randomly set, such as Group 1. Paclitaxel, cisplatin and Bevacizumab combination therapy (Bevacizumab, 5 mpk, i.p., q.w.*1+Cisplatin, 2.5 mpk, i.v., q.w.*1+Paclitaxel, 10 mpk,i.v., q.w.*1); Group 2. PD1 antibody (10 mpk, i.p., twice per week); Group 3. PD1 antibody combined with Bevacizumab (PD1 antibody, 10 mpk, i.p., twice per week+Bevacizumab, 5 mpk, i.p., q.w.*1); Group 4. PD1 antibody combined with cisplatin (PD1 antibody, 10 mpk, i.p., twice per week+Cisplatin, 2.5 mpk, i.v., q.w.*1), and the drugs were administered to each of the groups.

### 3. Cell Viability and Phenotype Detection

3-21 days after administration (day 10 here), the mice were euthanized, and the PVDF tubes were removed. Cell viability was quantified by the CellTiter-Glo (CTG) chemiluminescence method, and the relative ratio and basic phenotype of tumor cells and immune cells in PVDF tubes were detected by flow cytometry.

### 4. Data Analysis

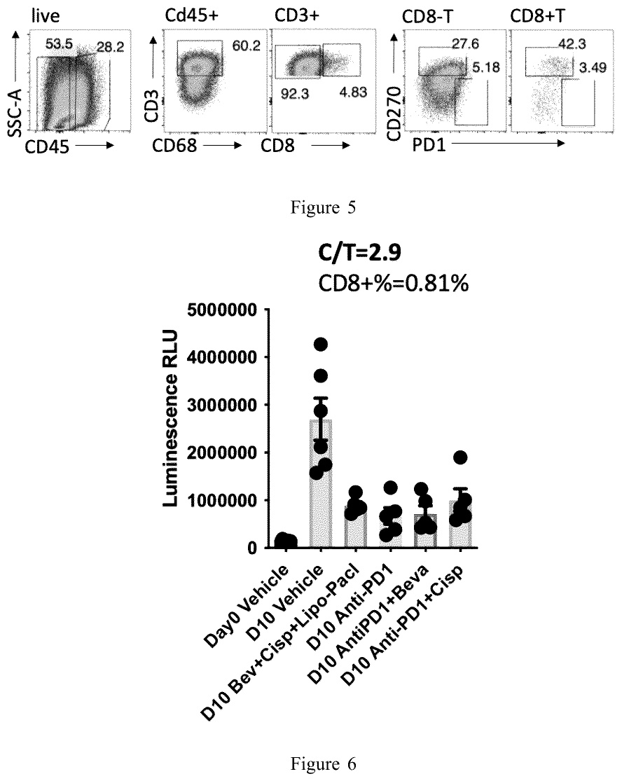

Flow cytometry (FACS) screening on day 0 was as follows: Phenotype analysis of primary tumor sample (tumor cells and immune cells mixed samples) in capsule tubes indicated that the ratio of CD45− tumor cells to CD3+ T cells was about 2.91:1, and the CD8+ T cell percentage was 0.81% out of total living cells. Tumor infiltrating T lymphocytes weakly expressed PD1, but, significantly, highly expressed CD270 (as shown in FIG. 5).

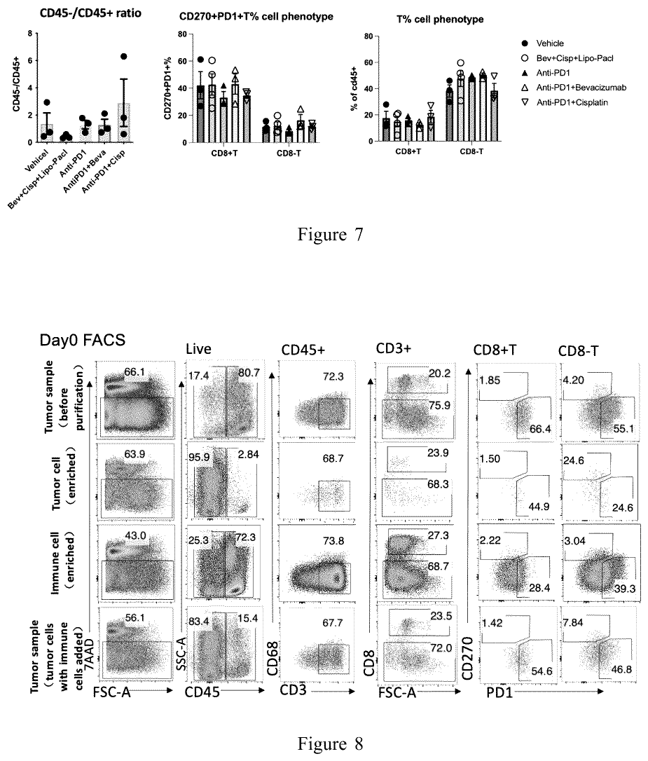

The endpoint of experiments in vivo on day 10 was as follows. According to the viability value of the cells in the control group (as shown in FIG. 6), the proliferation viability of all cells in the drug administration group was calculated, and then the effectiveness of the drug to be tested was calculated by CTG assay. The relative ratio and cell phenotype of tumor cells and immune cells in PVDF tubes were detected by flow cytometry (as shown in FIG. 7), analyzed by Flowjo software.

Based on the experimental results shown in FIGS. 6 and 7, at least half of the live cells in the capsule tubes on day 10 were CD45− tumor cells. Compared with the control group, PD1 monotherapy group and the other PD-1 combination therapies could significantly inhibit the overall viability of tumor tissues (including tumor cells and immune cells) without significantly changing the CD8+ T and CD8− T cell percentage. This data indicated that PD1 Ab monotherapy would be clinically better than first line Paclitaxel, cisplatin and Bevacizumab combination therapy based on the CTG data. CD270+ PD1+ double-positive TILs (tumor infiltrating lymphocytes) were enriched in the capsules along with the progression of the tumor, and the PD1 mono-therapy decreased this population, or blocked its PD1 molecule expression. Combined with the FACS data typing in Example 1, these data indicated that the CD270 molecule was efficiently expressed in solid tumors, which was a positive biomarker for the use of immune checkpoint inhibitor PD1 antibody therapy. Therefore, even patients with weak initial PD1 expression and significant CD270 expression would still respond well to immunotherapy.

Bioinformatic analysis of tumor cells and immune cells in PVDF capsule tubes of the clinical samples on day 0 or day 10 was detected by molecular biological methods (e.g., DNA sequencing, RNA sequencing and other “omics” technologies).

### Example 3 Rapid Functional Screening of Immunotherapy for a Patient with Ovarian Cancer

1. Experimental Animals, Sample Collection, and Cell Processing were the Same as in Example 1.

### 2. Flow Cytometry Detection and Data Analysis

The following was collected for cell phenotyping identification: a single cell suspension of patient tumor tissue samples before purification, tumor cells after purification, enriched immune cells (containing CD3+ T cells) eluted from the column, and a cell suspension in which tumor cells after purification and immune cells were mixed in a certain ratio (1.5:1 to 15:1), to yield a final cell count of 0.1 to 0.3 million cells. (Note that if the viability of immune cells eluted from the column was less than 20%, then downstream experiments and operations could not be performed, especially since activated immune cells were more likely to undergo activation-induced apoptosis). Then, each sample suspension was divided into multiple portions according to the purpose of the experiment, and the corresponding flow cytometry antibody mixture was added, and the corresponding experimental groups with compensation control were set up, including direct labeling or indirectly staining, and incubated at 2-8° C., 25-30 min in darkness for staining. The FACS panel for ovarian cancer were as follows: 7AAD, Antibodies to CD45, CD3, CD8, CD270, PD1, CD68 or CD25. After staining, the cells were rinsed with FACS buffer (PBS containing 2% FBS), then spun down at 600 g for 5 min at 4° C. The supernatant was removed, and the cell pellet was resuspended in 250 μl FACS buffer and loaded for flow cytometry detection. Data analysis for flow cytometry detection was performed by Flowjo software.

3. Cells within the Tubing
The basic operational steps were the same as those in Example 1.
According to the data analyzed by flow cytometry, the CD45− tumor cells and CD3+ T cells were mixed in a certain ratio (about 1.5:1-15:1). CD8+ T cells accounted for more than 0.5% of total live cells. The suspension was filled into PVDF tubes and sealed. In this example, the pre-purification tumor samples were also used for controls for tube sealing and other experiments.

### 4. Inoculation and Drug Administration

As above, the PVDF tubes were inoculated into mice subcutaneously with a puncture needle, and the wounds were sealed with medical tissue glue (available from 3M), and control groups and the drug administration groups were randomly set (Group 1. Paclitaxel, cisplatin and Bevacizumab combo-therapy, Group 2. PD1 antibody; group 3. PD1 antibody combined with PARP inhibitor Niraparib; Group 4. PARP inhibitor Niraparib), and the drugs were administered to each of the groups, similarly to Example 1.

### 5. Cell Viability and Phenotype Detection

3-21 days after administration (day 10 here), the mice were euthanized, and the PVDF tubes were removed. Cell viability was quantified by the CellTiter-Glo chemiluminescence method, and the relative ratio and basic phenotype of tumor cells and immune cells in PVDF tubes were detected by flow cytometry.

### 6. Data Analysis

Flow cytometry (FACS) screening on day 0 results for the relative ratio and cell phenotype of primary tumor cells and immune cells: (1) In the tumor tissue before purification, the ratio of CD45− cancer cells to CD3+T cells (C/T) was 0.3, and the CD8+T cell percentage was 11.5, which meant that TILs were rich in the tumor sample of this patient; (2) Tumor tissue cells after purification, contained only 2.84% immune cells, with a C/T value of 49.1, and CD8+cells were 0.448%; (3) The total viability of the immune cell population after purification was about 43%, and cells retained the basic cell phenotype as before purification. (4) Tumor tissue samples after adding immune cells had a C/T ratio of 8 and a CD8+percentage of 2.45%. The tumor infiltrating T lymphocytes weakly expressed CD270 molecules and highly expressed PD1 molecules, indicating this patient would have a good clinical response to PD1 Ab therapy (see FIG. 8).

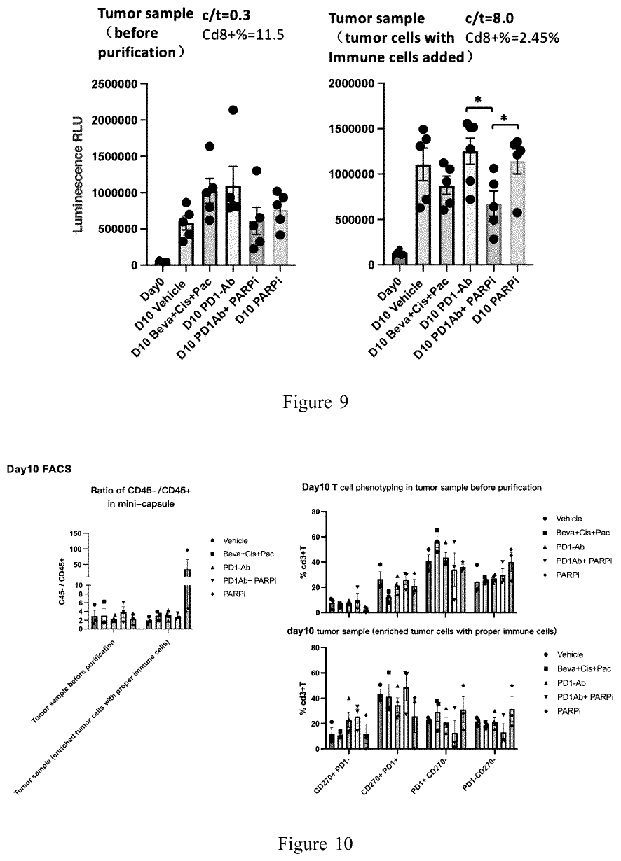

The endpoint of experiments in vivo on day 10 was as follows. Adjusted to the viability value of the cells in the control vehicle group (as shown in FIG. 9), the proliferation viability of all cells in the drug administration group was calculated, and then the efficacy of the drug to be tested was calculated. The relative ratio and cell phenotype of tumor cells and immune cells in PVDF tubes were detected by flow cytometry (as shown in FIG. 10), analyzed by Flowjo software.

Based on the experimental results shown in FIG. 9 and FIG. 10, the tumor samples tested on day 10 within the capsules, either without purification or with autologous TILs modification, were mainly CD45−(tumor) cells, which were present at about 2-3 times of the CD45+ cells (FIG. 10); CTG data of the tumor tissue sample before purification indicated that all the tested therapies, including the clinical first-line combo-therapy (Paclitaxel, cisplatin and Bevacizumab), would not significantly reduce the viability of total cells of the tissue sample. However, the response of the tumor sample after addition of autologous TIL cells, indicated a clinical first-line drug combination could weakly reduce the viability of total cells of the sample, consistently with the clinical evidence; although neither PD1 antibody nor PARP inhibitor mono-therapy could inhibit the overall tumor viability, their combination could significantly suppress the overall tumor proliferation and activity, better than clinical 1^{st} line chemical combo-therapy (FIG. 9). The combination of PD1 antibody and PARP inhibitor obviously increased the ratio of CD270+ PD1− T cell subgroup, meanwhile relatively reduced the percentage of PD1+ CD270− and PD1− CD270−T cells (FIG. 10), indicating that CD270 molecule is a biomarker for the effectiveness of PD1 antibody therapy. Notably, the patient had a poor clinical response to PARP inhibitor monotherapy, as predicted.

### Example 4 Rapid Functional Screening of Immunotherapy for a Patient with Lung Cancer

1. The Experimental Animals, Sample Collection and Cell Processing were the Same as in Example 1.

### 2. Flow Cytometry Detection and Data Analysis

A single cell suspension of the patient tumor tissue sample was made before purification. Then tumor cells after purification and enriched immune cells were collected respectively, and mixed in a certain ratio (about 1.5:1-15:1). The mixed cell suspension was divided into multiple portions according to the purpose of the experiment. The corresponding flow cytometry labeling antibodies were added, and the corresponding experimental groups, including the compensation controls, were set up by either direct or indirect labeling. The sample was stained at 2-8° C. for 25-30 min in darkness. The FACS panel for lung cancer included 7AAD, anti-CD45, anti-CD3, anti-CD8, etc. After completion of antibody labeling, PBS with 2% FBS was added and centrifuged at 600 g for 5 min at 4° C., and then the supernatant was removed. The cell pellet was resuspended in 250 μl FACS buffer (PBS+2% FBS) and loaded for flow cytometry detection. Data analysis for flow cytometry detection was performed by Flowjo software.

3. Cells within the Tubing

The capsule device was taken from the biological safety cabinet. The PVDF tube had a diameter of 1-2 mm, and the average pore size was about 500 KDa molecular weight. The PVDF tube was subjected to anhydrous ethanol activation, ultra-pure water flushing and autoclaving before the cell suspension was used to fill the capsule. A capsule tube of 1.5 cm in length was taken and washed repeatedly with cell culture medium (Gibco).

The tumor cells and CD3+ lymphocytes were mixed well to a certain ratio (1.5:1-15:1) based on the FACS validation. The CD8+T cells accounted for more than 0.5% of total living cells. The suspension was used to fill the PVDF tubes and sealed.

### 4. Inoculation and Drug Administration

The above PVDF tubes were inoculated into mice subcutaneously with a puncture needle, and the wounds were sealed with medical tissue glue (available from 3M). The control groups and drug administration groups were randomly set (such as Group 1. PD1 antibody (10 mpk, i.p., twice per week); Group 2. PD1 antibody combined with HDAC inhibitor Chidamide (30 mpk, p.o.,q.d.*7), and the drugs were administered.

### 5. Cell Viability and Phenotype Detection

3-21 days after administration (day 14 here), the mice were euthanized, and the PVDF tubes were removed. Cell viability was quantified by the CellTiter-Glo chemiluminescence method, and the relative ratio and basic phenotype of tumor cells and immune cells in PVDF tubes were detected by flow cytometry.

### 6. Data Analysis

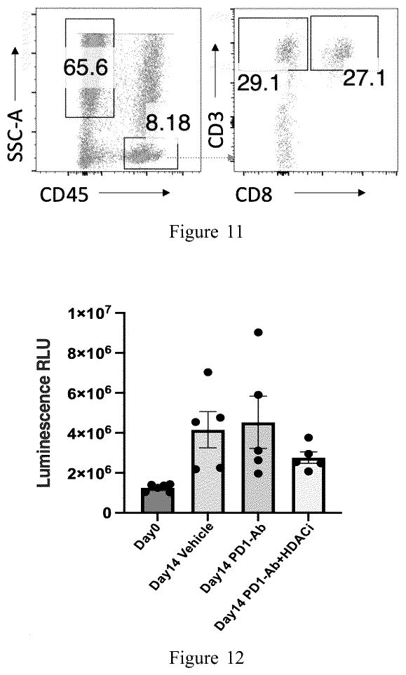

Flow cytometry (FACS) results on day 0 for phenotyping of patient tumor sample tested were as follows. Flow cytometry data of mixed cells in capsule tubes indicated that the ratio of CD45− tumor cells to CD3+ T immune cells was about 15:1, and in total live cells, CD8+% was 2.2% (as shown in FIG. 11).

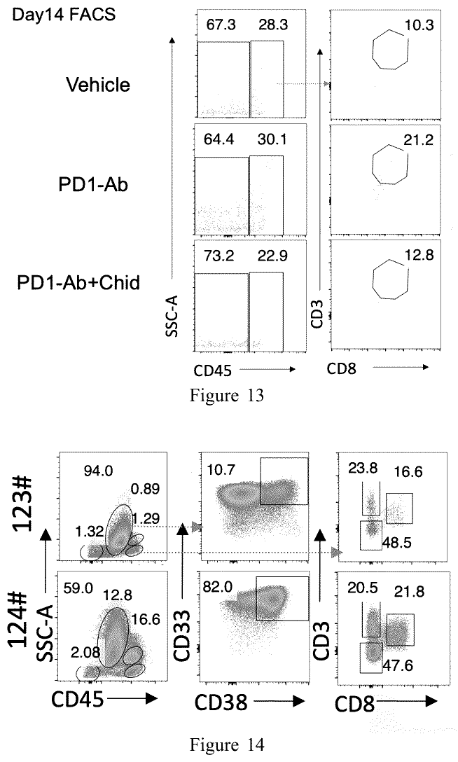

Experiment results in vivo on day 14 were are follows. Based on the viability value of the cells in the control group (as shown in FIG. 12), the proliferation and viability of all cells in the drug administration groups were calculated, as well as the efficacy of the drug by CTG assay. The relative ratio and cell phenotype of tumor cells and immune cells in PVDF tubes were detected by flow cytometry (as shown in FIG. 13), analyzed by Flowjo software.

Based on the experimental results shown in FIG. 12 and FIG. 13, CD45− tumor cells were dominant in the capsule tubes on day 14, at 60-75% (FIG. 13); PD1 antibody mono-therapy did not down-regulate the viability of total cells of tumor cells and immune cells (FIG. 12), although it promoted the survival of CD8+ T cells (in FIG. 13); When PD1 antibody was combined with HDAC inhibitor chidamide, the viability of total cells was clearly reduced, even though not reaching a statistical significance level (FIG. 12). Therefore, our data indicated this lung cancer patient was not suitable for PD1 Ab monotherapy, but combo-therapy of PD1 Ab and Chidamide would be recommended for clinical use for this patient.

### Example 5 Rapid Functional Screening and Potential Biomarkers Identification of Immunotherapy for Two Patients with Acute Myeloid Leukemia AML

### 1. Experimental Animals and Sample Collection

BALB/c Nude mice (available from Jiangsu Jicui Yaokang Biotechnology Co. Ltd.) aged 6-8 weeks were ordered from the supplier and kept in the animal house at SPF level. The animals were adaptively kept for at least 3 days before the start of the experiment.

Fresh peripheral blood samples from two acute myeloid leukemia (AML) patients (in the acute phase of AML) were collected and placed in anticoagulant tubes and transported to the central laboratory by cold chain (2-8° C.) within a short period of time.

### 2. Cell Processing

The peripheral blood samples of different patients were transferred into the biological safety cabinet. The cells were centrifuged using a gradient centrifugation gel (available from SIGMA) with a density of 1.077 g/mL at room temperature. Two peripheral blood samples of patients were respectively added into one of the centrifugal tubes, and the above-mentioned liquid was slowly placed into the centrifuge, with the temperature set at 20-25° C. (room temperature). Cells were centrifuged at 400 g for 20 min. at room temperature. After centrifugation, the middle cloudy cell layer (containing a large number of mononuclear cells) was collected, washed with 15 ml PBS (available from Gibco), and subjected to centrifugation at 1500 rpm for 5 min and the supernatant removed, and the cell suspension was subjected to further centrifugal washing. When necessary, 3-5× of erythrocyte lysis solution (available from Invitrogen) was used to lyse red blood cells. The cells were washed for 5 min at 1500 rpm, and the supernatant was removed. After resuspension in cell culture medium (available from Gibco), the cells were counted.

### 3. Flow Cytometry Detection and Data Analysis

Aliquots of 0.3 million peripheral blood cells were stained with different flow cytometry antibodies according to the experimental purpose, incubated 25-30 min at 2-8° C. in darkness for antibody labeling. The FACS panel commonly used in AML patients was: 7AAD, antibodies to CD45, CD3, CD8, CD33, CD38, CD20, CD25 or PD1. After staining, PBS with 2% FBS was added, and centrifuged at 600 g for 5 min at 4° C. and the supernatant was removed. The cell pellet was resuspended in 250 μl FACS buffer (PBS+2% FBS) and loaded into the flow cytometer. Data analysis for flow cytometry was performed using Flowjo software.

4. Cells within the Tubing

The capsule device was taken from the biological safety cabinet. The PVDF tube had a diameter of 1-2 mm (the average pore size of the material was about 300 kDa-700 kDa, allowing the ingress and egress of macromolecular proteins below the limitation of the PVDF permeability but not mammalian cells). PVDF tube was subjected to anhydrous ethanol activation, ultra-pure water flushing and autoclaving and the like before being filled with the cell suspension. A capsule tube of 1.5 cm in length was taken and washed repeatedly with cell culture medium (available from Gibco).

Quality control analysis was performed according to the flow cytometry analysis in step (3) so as to meet the requirement that the ratio of CD8+ T cells was at least more than 0.5% (the minimum value for effective measurement). Then, the tumor cells (CD45^{low/+} SSC-A^{mid/hi} CD33+/CD38+ cells) and the CD45^{high} SSC-A^{low}CD3+ T lymphocytes were uniformly mixed to a certain ratio (1.5:1 to 15:1) and then filled into PVDF tubes and sealed.

### 5. Inoculation and Drug Administration

The above PVDF tubes were inoculated into mice subcutaneously with a puncture needle, and the wounds were sealed with medical tissue glue (available from 3M). The control groups and drug administration groups were randomly set, such as PD1 antibody (Sintilimab or pembrolizumab, 10 mpk, i.p., twice per week), CD38 antibody Daratumumab (5 mpk, i.v., twice per week), Bc12 inhibitor Venetoclax (100 mpk, p.o. qd*6), HDAC inhibitor chidamide (30 mpk, p.o., q.d.*7), 5-Azacytidine (0.5 mpk, i.p., q.d.*5), in monotherapy or combo-therapy, and the drugs were administered to the groups.

### 6. Cell Viability and Phenotype Detection

3-21 days after administration (e.g., day 14 here), the mice were euthanized, and the PVDF tubes were removed. Cell viability was quantified by the CellTiter-Glo chemiluminescence method. Changes in the relative ratio and phenotype of tumor cells and immune cells in PVDF tubes were detected by flow cytometry. Bioinformatic changes of tumor cells and immune cells in PVDF capsule tubes of clinical samples at day 0 or day 10 were detected by molecular biological methods (RNA sequencing, and DNA sequencing technology, etc.).

### 7. Data Analysis

Flow cytometry results on day 0: Phenotyping analysis of primary tumor cells and immune cells for the two AML patients (#123, #124) are shown in FIG. 14. The CD8+ T cells accounted for 0.21% of all living cells and C/T (the ratio of CD45^{low}CD33+CD38+ cancer cells to CD3+T) was about 15:1 in patient #123 (and more CD38− tumor cells were present in patient 123 #), while the CD8+ T cell percentage was about 3.48% and the C/T value was about 6:1 in patient #124.

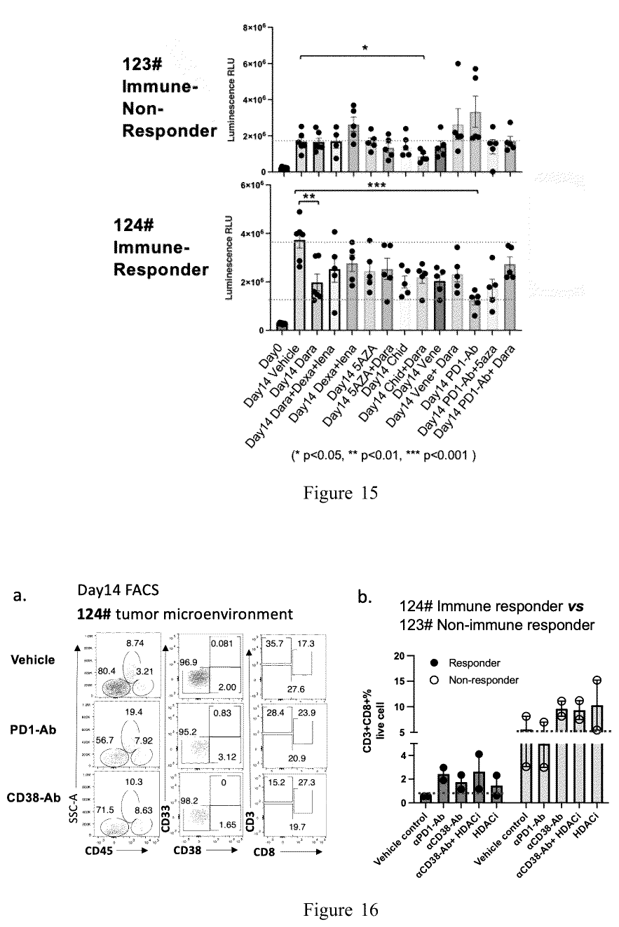

Cell viability as measured by luminescence (CTG method) on day 14: according to the viability value of the cells in the control group (as shown in FIG. 15), the proliferation viability of all the cells in the drug administration group was calculated, and then the efficacy of the drug was calculated. Flow cytometry detection on day14: the relative ratio of tumor cells and immune cells and their phenotyping in PVDF tubes was analyzed by flow cytometry (as shown in FIG. 16).
Based on the above data analysis of cell viability as measured by luminescence (CTG method) and FACS on day 14, the vast majority of living cells in the capsule were CD45−/low tumor cells (AML cancer blasts), and around 5%-10% were CD45^{high} SSC-A^{low} lymphocyte population (#124 as an example shown in FIG. 16a). Patient #124 (“Immune Responder” because the patient's T cells are responding to PD1 antibodies) showed a positive response to the checkpoint inhibitor PD1 antibody as more than half of inhibitory effect on cell viability was identified in PD1 antibody group compared with control vehicle (in FIG. 15), which coincided with significant reduction of AML blasts in living cells on day14 (FIG. 16a), as well as increase of CD3+ CD8+ (FIG. 16b). This phenomenon did not appear in patient #123 (“Immune Non-responder” because the patient's T cells are not responding to PD1 antibodies). The #123 patient had a poor clinical response to Venetoclax. In addition, CD38 antibodies operated with an antibody-dependent cytotoxicity (ADCC) function and antibody-dependent macrophage phagocytosis (ADCP) function. The data also showed such reactivity in this system, as patient #124 showed great response to CD38 antibody therapy, which was closely related to the high expression of CD38 antigen. Meanwhile, CD38 was weakly expressed in patient #123, only 10.7% and this patient did not respond to CD38-Ab monotherapy as predicted (FIG. 14). Therefore, the activity and function of NK cells and mononuclear macrophages (both were CD45+) was also retained during the collection of immune cells on day 0 in this system.

### 8. Bioinformatics Analysis

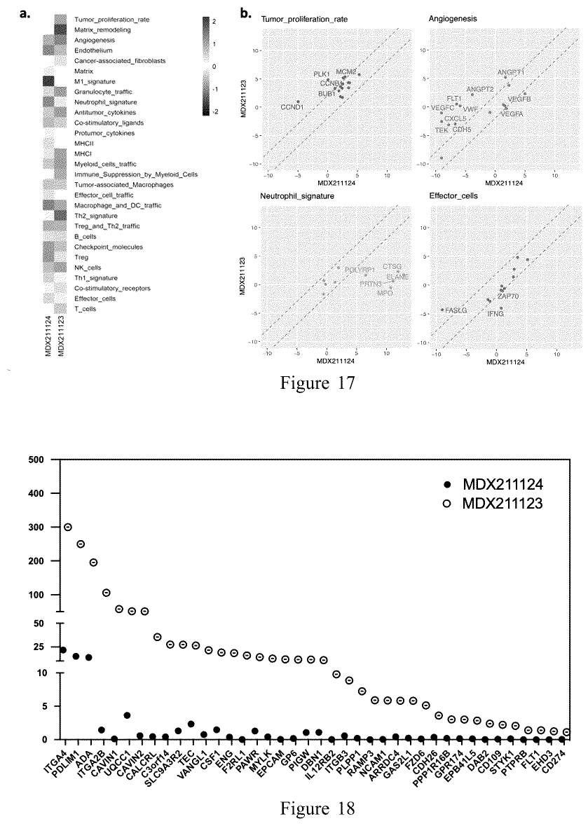

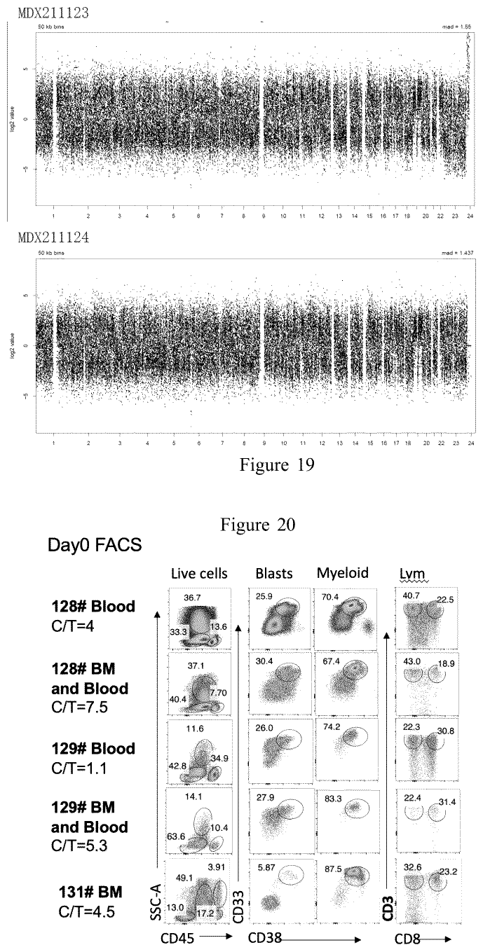

The bioinformatic fingerprint of overall tumor cells and immune cells of the patient cells in PVDF capsule tubes on day 0 was detected by molecular biological methods (RNA-seq and DNA-seq) so as to identify the biomarkers that may be reactive with immune drugs for the patient and reveal the molecular pathophysiological basis of disease occurrence and development.
RNA-seq analysis of transcriptome differences between two patients:
(1) Analysis of differential gene signaling pathways: as shown in FIG. 17a and FIG. 17b, the immune non-responder patient #123 had high tumor proliferation activity (such as CCDN1, PLK1, MCM2), strong ability to remodel tumor microenvironment, high expression of neovascular associated markers (such as VEGFC, ANGPT2, FLT1, VWF), and low function of T cells, especially effector T cells. Immune responder patient #124 had low proliferation of tumor itself and low angiogenesis ability, but relatively powerful effector function of T cells (such as IFNγ producing) and neutrophils (such as ELANE, MPO, PRTN3).
(2) Potential key biomarkers identification for immunotherapy in AML patients (subsets of membrane protein): as shown in FIG. 18, the analysis of differential expression of membrane proteins indicated that compared with #124 immune responder patient, #123 immune non-responder patient significantly expressed higher levels of oncogenes or cancer susceptibility genes (ITGA4, ITGA 2B, ITGB3, PDLIM1, ADA, CAVIN1, CAVIN2, C3orf14, TEC, EPCAM, CD109, etc.), immune suppression related genes (CSF1, CD274/PDL1) and other membrane proteins that promote the occurrence and development of tumors, as well as showing expression of novel tumor targeting molecules that have not yet been reported, including SLC9A3R2 (SLC9A3 regulator 2), PIGW (phosphatidylinositol glycan anchor biosynthesis class W), CDH26 (cadherin 26) and PPP1R16B (protein phosphatase 1 regulatory subunit 16B). These four membrane molecules are not only the biomarkers of hematologic tumors, but also potential targets for solid cancers (pan-cancers) from the TCGA database.
   DNA-seq analysis of genomic differences between two AML patients:
   As shown in FIG. 19, the WES-CNV signal varied greatly, and there was no apparent deletion and amplification. Well-known biomarkers related to AML were not found, including fusion genes (ABL1-BCR, CRLF2, JAK2-BRAF, NOTCH1), mutant genes (FBXW7, JAK1, KRAS, NOTCH1, NPM1, FLT3, KIT, CEBPA), etc. That is to say, no key mutations of the reported genes were found in the two patients at the genome level.

### Example 6 Rapid Functional Screening and Potential Biomarker Identification of Immunotherapy for Three Patients with Acute Myeloid Leukemia AML

### 1. Experimental Animal Preparation was the Same as in Example 5.

### 2. Sample Collection

Fresh peripheral blood samples and/or bone marrow from three acute myeloid leukemia (AML) patients (in the acute phase) were collected and placed in anticoagulant tubes, and transported to the central laboratory by cold chain (2-8° C.) within a short period of time.

### 3. Sample Processing and Flow Cytometry Detection of the Cells was the Same as in Example 5.

4. Cells within the Tubing

Similar to Example 4, the bone marrow samples and blood samples of some patients were mixed at 4:1 to 5:1 to ensure that the tumor tissue samples contained circulating peripheral blood cells so as to maximally mimic the function of the human microenvironment. Quality control analysis was performed according to the data obtained by flow cytometry so as to meet the requirement that the ratio of CD8+ T cells was at least more than 0.5% (the required minimum concentration), and the tumor cells (CD45^{low/+} SSC-A^{mid/hi} CD33+/CD38+ cells) and the CD45^{high} SSC-A^{low}CD3+ T lymphocytes were uniformly mixed according to a certain ratio (C/T (CD38+Cancer/CD3+T)=1.5:1 to 15:1), and then filled into PVDF tubes and sealed.

### 5. Inoculation and Drug Administration

The same as Example 4, the control groups and drug administration groups were randomly set, such as CD38 antibody (Daratumumab, 5 mpk, i.v., twice per week), CD38 antibody combined with PD1 antibody, PD1 antibody combined with decitabine (0.5 mpk, i.p., q.d.*5), PD1 antibody alone (Sintilimab or pembrolizumab, 10 mpk, i.p, twice per week), and the drugs were administered to the mice.

### 6. Data Analysis

Flow cytometry sorting on day 0: as shown in FIG. 20, the blood C/T (CD38+ cancer/CD3+ T cell ratio) of patient #128 was 4, and the C/T ratio of mixed bone marrow and blood sample was 7.5; the blood C/T (CD38+ cancer/CD3+ T cell ratio) of patient #129 was 1.1 (and more cancer blasts did not express CD38, that is to say, the C/T value of the patient was actually greater than 1.5), and the CT of mixed bone marrow and blood sample was 5.3. The C/T bone marrow sample of patient #131 was 4.5. The myeloid cancer cells of these three patients all highly expressed CD38 molecules (about 70%-80%), and CD8+ T cell percentage was also between 1% and 10%.

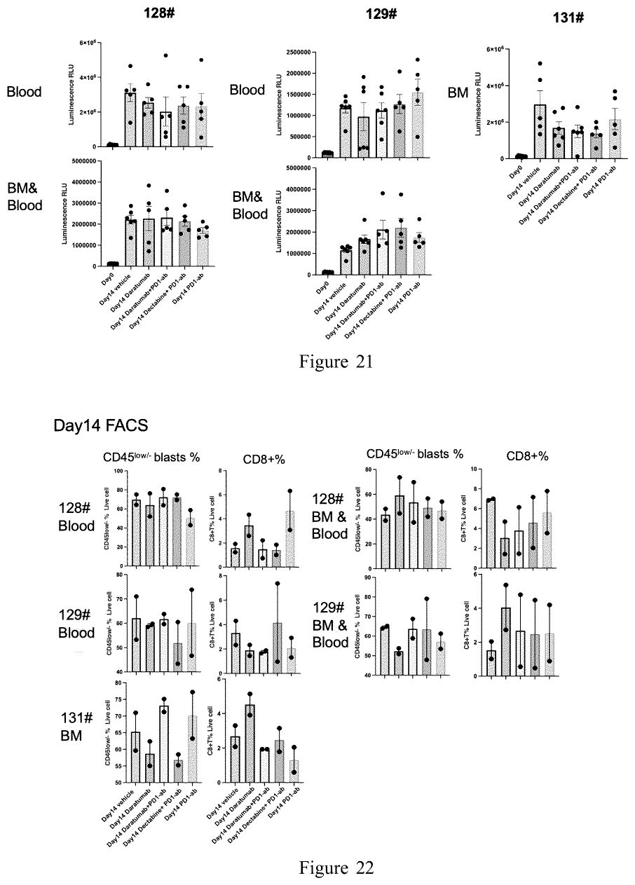

Cell viability as measured by luminescence (CTG method) on day 14: according to the viability value of the cells in the control group (as shown in FIG. 21), the proliferation viability of all the cells in the drug administration group was calculated, and then the efficacy of the drug was calculated.

Flow cytometry detection on day14: the relative ratio of tumor cells and immune cells in PVDF tubes was detected by flow cytometry. The differences between tumor cells and immune cells in the drug group and the control group were compared (as shown in FIG. 22). The results were analyzed by Flowjo software.

According to the results shown in FIG. 20, FIG. 21, and FIG. 22, although all three patients highly expressed CD38 target protein, patients #128 and #129 hardly had a response effect to CD38 antibody as the total number of living cells did not decrease after drug treatment (FIG. 21), and cancer blasts still occupied a dominant proportion in the capsules (FIG. 22). Patient #131 had a positive response to CD38 Ab mono-therapy, CD38 Ab with PD1 Ab combo-therapy, as well as decitabine with PD1 Ab combo-therapy as predicted (FIG. 21); less AML blasts and more CD8+ TILs were identified after CD38 Ab mono-therapy, or PD1 Ab with decitabine combo-therapy (FIG. 22).

The overall bioinformatic fingerprint of tumor cells and immune cells of the patients in PVDF capsule tubes on day 0 or day 14 was detected by molecular biological methods (RNA-seq and DNA-seq) so as to identify and evaluate the biomarkers of the patient's sensitivity and resistance to immune drugs.

### Example 7 Rapid Functional Screening of Immunotherapy for a Patient with Acute Myeloid Leukemia AML

1. Experimental Animals were the Same as in Example 5.

### 2. Sample Collection

Fresh peripheral blood and bone marrow samples from an AML patient (in the acute phase of AML) were collected, and placed in anticoagulant tubes, and transported to the central laboratory by cold chain (2-8° C.) within a short period of time.
3. Cell Processing, Flow Cytometry Detection, and Data Analysis were the Same as in Example 5.
4. Cells within the Tubing
Similar to Example 6, the bone marrow sample and blood sample of the patient were mixed at 4:1 to 5:1 to ensure the tumor tissue sample contained circulating peripheral blood cells so as to maximally mimic the functional human microenvironment. Quality control analysis was performed according to the data of flow cytometry, it was found that the percentage of CD8+T was 0.466% (close to the threshold value of 0.5%), and the ratio of tumor cells (CD38+CD33+tumor cells) and CD45^{high} SSC-A^{low}CD3+T was 8 (belonging to the range of C/T=1.5:1 to 15:1). Downstream exploratory experiments were also performed, and the samples were filled into PVDF tubes and sealed.

### 5. Inoculation and Administration

Similar to Example 5, the control groups and different administration groups were randomly set up (such as bc12 inhibitor Venetoclax, CD38 antibody, CD38 antibody combined with Chidamide, CD38 antibody combined with 5AZA, CD38 antibody combined with Venetoclax, PD1 antibody, PD1 antibody combined with SAZA, etc.), and the drugs were administered.

6. Cell Viability and Phenotype Detection were the Same as in Example 5.

### 7. Data Analysis

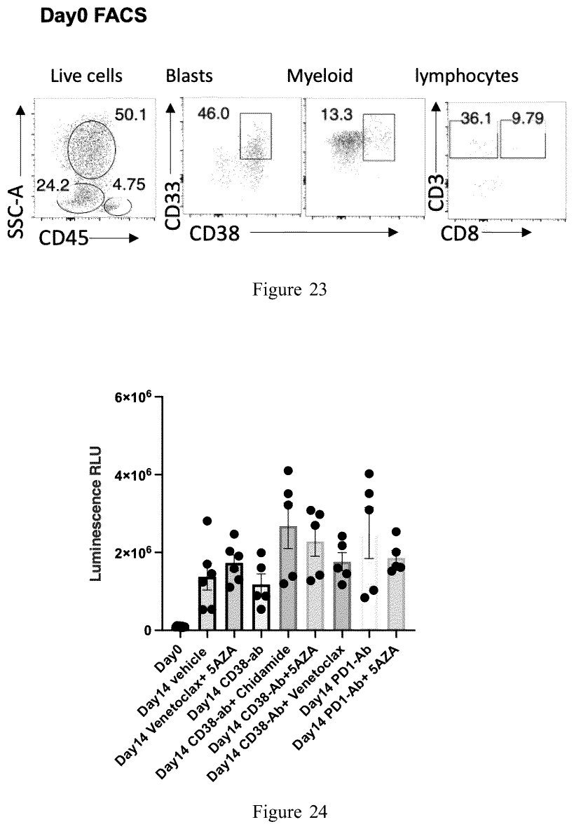

Flow cytometry (FACS) sorting on day 0: as shown in FIG. 23, C/T was 8 (since more tumor cells did not express CD38, that is to say, the ratio of cancer cells to CD3+ T cells (C/T) was actually much larger than 8) for the mixed sample of bone marrow and blood of patient #160, and the CD8+ T cell percentage was 0.466%.

Cell viability as measured by luminescence (CTG method) on day 14: according to the viability value of the cells in the control group (as shown in FIG. 24), the proliferation viability of all the cells in the drug administration group was calculated, and then the efficacy of the drug was calculated.

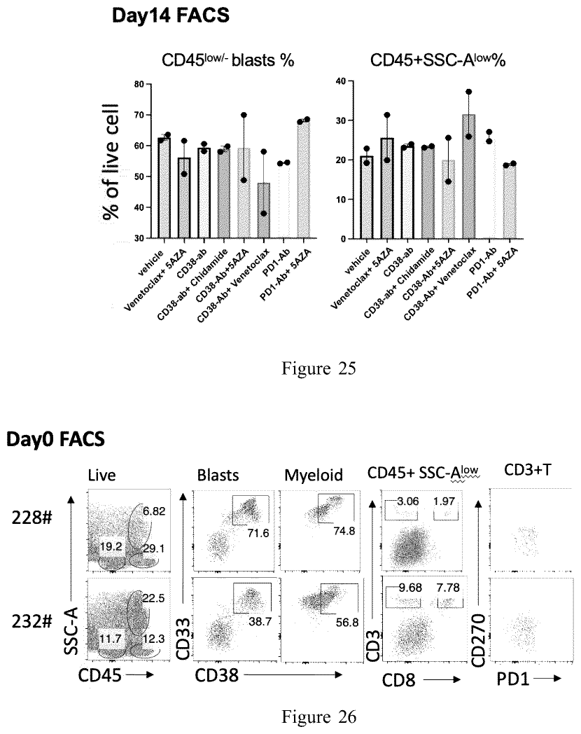

Flow cytometry detection on day 14: the relative ratio of tumor cells and immune cells in PVDF tubes was detected by flow cytometry; the differences between tumor cells and immune cells in the drug group and the control group were compared (as shown in FIG. 25). The results were analyzed by Flowjo software.

According to the results of flow cytometry and cell viability as measured by CTG method on day 14, CD45^{low/-} tumor precursor/progenitor cells were predominant in capsules, and the lymphocyte population accounted for about 20%. None of the tested drugs could significantly inhibit the proliferation of the tumor cell population. This experiment showed that a high C/T ratio (>8) and a low CD8+% (<0.5%) results in a non-response to immunotherapy for this patient. Therefore, the clinician should not prescribe PD-1 targeting therapy or CD38 antibody therapy for the patient.

### Example 8 Rapid Functional Screening of Immunotherapy for Multiple (Two) AML Patients with Acute Myeloid Leukemia Wherein the Same Mouse was Inoculated

1. Experimental Animals were the Same as in Example 5.

### 2. Sample Collection

Fresh bone marrow samples from two AML patients (in the acute phase of AML) were collected and placed in anticoagulant tubes and transported to the central laboratory by cold chain (2-8° C.) within a short period of time.

3. Cell Processing, Flow Cytometry Detection and Data Analysis, and Placement of Cells within the Tubing were the Same as in Example 5.

### 4. Inoculation and Drug Administration

The capsules of bone marrow cells of two patients inoculated in the same mouse subcutaneously on both sides of the back of the mouse at 3 capsule tubes for each patient (two patients in one mouse). The mice were randomly grouped into control groups and the drug administration groups (such as CD38 antibody and PD1 antibody), and the mice in the administration group were administered the drug. At the same time, independent control and administration experiments (one patient in one mouse) were respectively carried out by this method, and a part of cells in the capsule tubes on day 10 were preserved and collected for FACS detection.

5. Cell Viability and Phenotype Detection were the Same as in Example 5.

### 6. Data Analysis

Flow cytometry sorting on day 0: as shown in FIG. 26, for #228 patient, bone marrow C/T (CD38+Cancer/CD3+T) was 12, and the CD8+ cell percentage was 0.57 (higher C/T value and low CD8+proportion, which show that the patient will not respond to T cell immunotherapy); for patient #232, the bone marrow C/T ratio was 8.5, and the CD8+ cell percentage was 0.85%. The proportion of CD3+T in both patients was low, and the expressions of PD1 and CD270 markers were also very weak, showing that PD1 antibody therapy for the patients will not be efficacious.

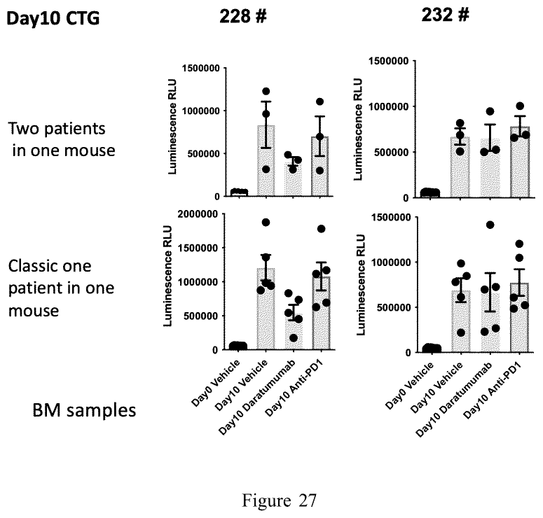

Cell viability as measured by luminescence (CTG method) on day 10: according to the viability value of the cells in the control group (as shown in FIG. 27), the proliferation viability of all the cells in the drug administration group was calculated, and then the efficacy of the drug was calculated.

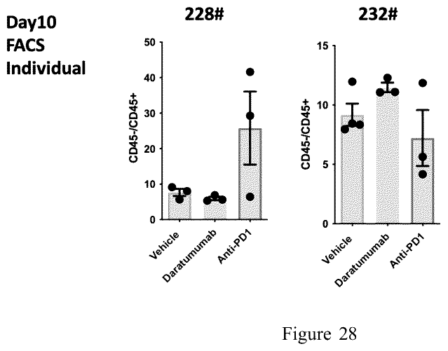

Flow cytometry detection on day 10: the relative ratio of tumor cells and immune cells in PVDF tubes was detected by flow cytometry; the differences between tumor cells and immune cells in the drug group and the control group were compared (as shown in FIG. 28). The results were analyzed by Flowjo software.

According to the results of cell viability as measured by CTG method and FACS on day 10, CD45− tumor (blast) cells were predominant in the capsules of the two patients (as shown in FIG. 28). AML patient #228 was sensitive to CD38 antibody therapy as the total cell viability was inhibited by about half (FIG. 27), and the ratio of CD45−tumor cells and CD45+immune cells was also slightly reduced compared with the control group (FIG. 28), while the other CD38⁺ patient #232 had no significant reactivity to CD38 antibody (FIG. 26 and FIG. 27). The reason for this difference in response might be due to the difference in the density of CD38 tumor antigen of patients and the structure and functional difference of bone marrow niche, such as the function of myeloid immune cells, e.g., NK, monocyte/macrophage, and neutrophils, with different antibody-dependent cytotoxicity (ADCC) and antibody-dependent macrophage phagocytosis (ADCP) abilities. Nevertheless, it was consistent with the earlier prediction that both patients would not respond to PD1 antibodies, possibly due to the fact that very few PD1+ CD3+ T cells existed in the tumor microenvironment of patient samples(FIGS. 26 and 27). In addition, the data of drug susceptibility detection of “two patients in one mouse” model was also consistent with the data of a traditional model of “one patient in one mouse” (FIG. 27). Therefore, for the rapid screening of efficacy of immune drugs in vivo, the same mouse can be used to load capsules of multiple patients for testing in order to save costs and resources, which can be distinguished and traced according to different inoculation sites (the present experiment), or size of different capsules, or even different color coding of the capsules, etc.

The important significance of the immunological drug functional screening method in vivo is summarized:
(1) The data in Example 1, Example 2, and Example 3 show that the method was useful for determining that some ovarian cancer patients rich in TIL with weak initial PD1/PDL1 expression would still have a positive response to PD1 antibody therapy. This is the first time any screening method has been demonstrated to show such a precise and unexpected prediction for how to use of PD1 therapy in clinical treatment of ovarian cancer. Significant or differential changes of CD270 expression in tumor-infiltrating lymphocytes were also shown to be a biomarker for PD1 antibody therapy. Even when the PD1 expression was abundant in tumor tissue, if the ratio and abundance of tumor cells and immune cells were unbalanced, the response of immunotherapy can also be affected, and the combination of immunotherapy and targeting therapy (or chemical therapy) may still be an effective way to overcome the non-response of mono-therapy (these data are also supported by Example 4).
(2) The data in Example 5, Example 6, and Example 7 show that the method can effectively screen and distinguish differential responses to CD38 or PD1 antibodies in vivo between CD38+/high and (or) TIL enriched patients with blood tumor (such as AML). In addition to the discovery of well-known cancer-driver genes or cancer susceptibility genes (these reported genes accounted for about more than 90% of the overall differential genes of membrane molecules that we analyzed, separately proving the reliability of the model), and new potential diagnostic and therapeutic targets have been found: SLC9A3R2 (SLC9A3 regulator 2), PIGW (phosphatidylinositol glycan anchor biosynthesis class W), CDH26 (cadherin 26) and PPP1R16B (protein phosphatase 1 regulatory subunit 16B). Therefore, this rapid-functional experiment-guided omics analysis was conducive to the discovery and research of new targets for the next generation of cancer therapy.
(3) Example 8 shows that the same mouse can be loaded with tumor capsules from multiple patients with the same disease for rapid efficacy screening of immunological drugs in vivo, and the results were proved to be consistent with the drug sensitivity experiment of traditional immunodeficiency mice loaded with tumor capsules from one patient, which was beneficial for cost saving and efficiency improvement.

I claim:

1. A method of treating a cancer patient with an immunomodulatory drug comprising the steps of:
a) isolating tumor cells and immune cells from the patient into a single-cell suspension,
b) combining the patient's tumor cells with autologous patient CD3+ lymphocytes to form a combination of total cells in an implantable capsule,
c) implanting the capsule in an immunodeficient mouse for a time,
d) administering an immunomodulatory drug to the mouse during this time,
e) removing the capsule from the mouse and analyzing the cell viability of the tumor, and
f) administering the immunomodulatory drug to the patient upon the condition that the immunomodulatory drug decreases the cell viability of the tumor in the capsule or increases the ratio of lymphocytes in the capsule.

2. The method of claim 1, wherein the immunomodulatory drug is anti-PD-1 or anti-CTLA 4.

3. The method of claim 1, further wherein the patient's tumor regresses.

4. The method of claim 1, wherein the immunomodulatory drug is optimal for treatment among a choice of other drugs for treating the patient's cancer.

5. The method of claim 1, wherein timing from removing the capsule to administration of the immunomodulatory agent in a cancer patient is two days, three days, four days, five days, six days, seven days, eight days, nine days, ten, eleven, twelve, thirteen, or fourteen days.

6. The method of claim 1, wherein the patient's tumor cells are combined with autologous patient CD3+T lymphocytes within a critical range of 1.5:1 to 15:1.

7. The method of claim 5, wherein the patient's tumor cells with autologous patient CD3+T lymphocytes are combined within a range of 1.5:1 to 9:1.

8. The method of claim 5, wherein the CD3+ lymphocytes comprise lymphocytes that co-express CD8.

9. The method of claim 8, wherein the combination of total cells has a total number of living cells and the ratio of CD8+ CD3+ lymphocytes to total live cells is 0.5% to 15%.

10. The method of claim 1, wherein the mouse has additional capsules implanted wherein the additional capsules comprise cells from the same patient or additional patients.

11. The method of claim 1, further wherein the immunomodulatory drug is combined with a second drug.

12. The method of claim 11, wherein the second drug is a different immunomodulatory drug, a targeted therapy drug, or a chemotherapeutic drug.

13. The method of claim 1, wherein an average protein permeability of the capsule tube is around 300-1,000 Kda, or 500-700 Kda.

14. The method of claim 1, wherein patient tumor cells and patient immune cells are in a single cell suspension, and further wherein the tumor cells and immune cells are derived from fresh tumor tissues or body fluids of a clinical patient.

15. The method of claim 14, wherein the tumor cells comprise clinically surgically resected tumor tissues or biopsied tumor tissues.

16. The method of claim 14, wherein the body fluids comprise one or more of blood, bone marrow, pleural fluid, and ascites, and cerebrospinal fluid.

17. The method of claim 14, wherein the lymphocytes are isolated from the peripheral blood mononuclear cells in peripheral blood of the patient.

18. The method of claim 14, wherein patient lymphocytes are enriched by sorting into a single cell suspension, and the patient tumor cells are those that flow through when sorting the single cell suspension, wherein the single cell suspension comprises tumor-infiltrating lymphocytes that infiltrated a solid tumor tissue of the patient.

19. The method of claim 14, wherein the sorting method used in said sorting comprises sorting cells by adherence to magnetic beads conjugated to anti-human CD45, sorting by flow cytometry or by using combination thereof.

20. The method of claim 19, wherein the viability of CD45+ immune cells is greater than 20%.
